(19) United States
(12) Patent Application Publication
(10) Pub. No.: US 2009/0214510 A1
(43) Pub. Date: Aug. 27, 2009

(54) Broad Spectrum Antiviral Compositions

(75) Inventors: Gary J. Nabel, Washington, DC (US); Jae Ouk Kim, Rockville, MD (US)

Correspondence Address: EDWARDS ANGELL PALMER & DODGE LLP, PO BOX 55874, BOSTON, MA, 02205, US

(73) Assignee: Government of the US as represented by the Secretary, Department of Health and Human Services, Rockville, MD (US)

(21) Appl. No.: 12/224,273

(22) PCT Filed: Feb. 21, 2007
(86) PCT No.: PCT/US07/04471

§ 371 Date: Nov. 21, 2008

Related U.S. Application Data
(60) Provisional application No. 60/775,666, filed on Feb. 21, 2006.

(51) Int. Cl. A61K 38/46; C12N 9/16; C07H 21/04; C12N 15/63; C12N 5/00

(52) U.S. Cl. 424/94.6; 435/196; 536/23.2; 435/320.1; 435/325

(57) ABSTRACT

The instant invention provides compositions and methods for the treatment of viral infections caused by enveloped viruses comprising phospholipase nucleic acid molecules or polypeptides, or fusion molecules comprising phospholipase molecules or functional fragments thereof.

### RELATED APPLICATIONS

This application claims the benefit of U.S. Provisional Application No. 60/775,666, filed Feb. 21, 2006, the entire contents of which are expressly incorporated herein by reference.

### GOVERNMENT SUPPORT

Research supporting this application was carried out by the United States of America as represented by the Secretary, Department of Health and Human Services.

### BACKGROUND OF THE INVENTION

Viral infection is an increasing clinical problem. Often clinicians find themselves in the position of diagnosing a viral infection in a subject and not having effective antiviral compositions to treat the infected subject. Various antiviral compounds have been designed for use to treat viral infections in humans. However, many of these compounds are virus specific, or restricted to particular strains of a given virus. Development of compounds which are effective at treating viral diseases caused by many different viral families has only recently become a major research focus.

Phospholipases are a family of enzymes that catalyze the conversion of phospholipids into fatty acids and other lipophilic substances. Four families of phospholipases have been identified and are designated A, B, C, and D. Previous studies have shown that phospholipase A2 is capable of inhibiting viral replication, but that biological activity, e.g., enzymatic activity, is not required for this antiviral activity (Fenard et al. (2001) Mol. Pharna. 60:34147 and Fenard et al. (1999) J. Clin. Invest. 104:611-18). This work demonstrates that antiviral activity of phospholipase A2 depends upon the secreted phospholipase, e.g., phospholipase A2, binding to cells and blocking viral entry into these cells.

There is a need in the field for novel antiviral compounds which are effective against a broad spectrum of viruses. Accordingly, the instant invention provides compositions and methods for the treatment of viral infection.

### SUMMARY OF THE INVENTION

The instant invention provides compositions and methods for the treatment of viral infection. The compositions of the invention are targeted phospholipase polypeptides comprising a biologically active, e.g., an enzymatically active, phospholipase, or biologically active fragment thereof, attached to a viral binding polypeptide, e.g., a polypeptide that recognizes a viral polypeptide or a carbohydrate, and optionally containing a linker.

Accordingly, in one aspect, the invention provides a polypeptide comprising a phospholipase polypeptide, or biologically active fragment thereof, and a viral binding polypeptide. In one embodiment, the phospholipase polypeptide, or biologically active fragment thereof, and a viral binding polypeptide are connected by a linker, e.g., a polypeptide linker.

In one embodiment, the phospholipase polypeptide is a mammalian, e.g., a human, phospholipase. In a related embodiment, the phospholipase polypeptide, or biologically active fragment thereof, is a phospholipase A polypeptide, or biologically active fragment thereof. In a specific embodiment, the phospholipase A polypeptide, or biologically active fragment thereof, is a phospholipase A2 polypeptide, or biologically active fragment thereof. In another specific embodiment, the phospholipase A2 polypeptide, or biologically active fragment thereof, comprises the phospholipase A2 polypeptide, as set forth in SEQ ID NO:1, or a biologically active fragment thereof.

In a related embodiment, the phospholipase polypeptide, or biologically active fragment thereof, consists of a polypeptide that is at least 90% identical to phospholipase A2 polypeptide, as set forth in SEQ ID NO:1 or a biologically active fragment thereof.

In another embodiment, the viral binding polypeptide binds to an enveloped virus, e.g., to a viral coat protein or a carbohydrate on an enveloped virus. Exemplary enveloped viruses include those belonging to Herpesviridae, e.g., herpes and CMV; Poxyiridae, e.g., variola and smallpox; Hepadnaviridae, e.g., hepatitis B virus; Togaviridae, e.g., Rubella; Flaviviridae, e.g., hepatitis C virus and yellow fever virus; Coronaviridae, e.g., SARS; Paramyxoviridae, e.g., PIV, RSV and measles; Bunyaviridae, e.g., Hantavirus; Rhabdoviridae, e.g., VSV and rabies; Filoviridae, e.g., Ebola, and Marburg; Orthomyxoviridae, e.g., influenza; Arenaviridae, e.g., Lassa; and Retroviridae, e.g., HIV and HTLV. In specific embodiments, the viral binding polypeptide binds to a viral coat protein from HIV, Amphovirus, Marburg virus, Dengue virus, Ebola virus and SARS virus. In a related embodiment, the viral coat protein is a glycoprotein, e.g., HIV gp120, SIV gp120, Ebola GP, Cytomegalovirus gB, Hepatitis C virus E1, Hepatitis C virus E2, and Dengue virus gE. In a specific embodiment, the viral coat protein is HIV gp120.

In one embodiment, the viral binding polypeptide is a DC-SIGN polypeptide, e.g., the DC-SIGN polypeptide as set forth in SEQ ID NO:3, or a biologically active fragment thereof. In a related embodiment, the DC-SIGN polypeptide is at least 90% identical to the sequence set forth as SEQ ID NO:3.

In one embodiment, the polypeptide linker is comprised of glycine and serine amino acid residues. In a specific embodiment, the polypeptide linker has the sequence (GlyGlyGlySer)₄.

In a specific embodiment, the invention provides polypeptide comprising a phospholipase A2, or a biologically active fragment thereof, and DC-SIGN polypeptide connected by a peptide linker. In a related embodiment, the polypeptide has the sequence as set forth in SEQ ID NO:5.

In one aspect, the invention provides a polynucleotide encoding a polypeptide comprising a phospholipase polypeptide, or biologically active fragment thereof, and a viral binding polypeptide, or biologically active fragment thereof. In a related embodiment, the polynucleotide encodes a polypeptide that further comprises a polypeptide linker. In a related embodiment the phospholipase polypeptide is a mammalian, e.g., a human, phospholipase.

In a related embodiment, the phospholipase polypeptide, or biologically active fragment thereof, is a phospholipase A polypeptide, or biologically active fragment thereof. In a related embodiment, the phospholipase polypeptide, or biologically active fragment thereof, is a phospholipase A polypeptide, or biologically active fragment thereof. In a specific embodiment, the phospholipase A polypeptide, or biologically active fragment thereof, is a phospholipase A2 polypeptide, or biologically active fragment thereof. In another specific embodiment, the phospholipase A2 polypeptide, or biologically active fragment thereof, comprises the phospholipase A2 polypeptide, as set forth in SEQ ID NO:1, or a biologically active fragment thereof.

In a related embodiment, the phospholipase polypeptide, or biologically active fragment thereof, is encoded by a polynucleotide that is at least 90% identical to a phospholipase A2 polynucleotide, as set forth in SEQ ID NO:2 or a fragment thereof that encodes a biologically active polypeptide.

In another embodiment, the viral binding polypeptide binds to an enveloped virus, e.g., to a viral coat protein on an enveloped virus. Exemplary enveloped viruses include those belonging to Herpesviridae, e.g., herpes and CMV; Poxyiridae, e.g., variola and smallpox; Hepadnaviridae, e.g., hepatitis B virus; Togaviridae, e.g., Rubella; Flaviviridae, e.g., hepatitis C virus and yellow fever virus; Coronaviridae, e.g., SARS; Paramyxoviridae, e.g., PIV, RSV and measles; Bunyaviridae, e.g., Hantavirus; Rhabdoviridae, e.g., VSV and rabies; Filoviridae, e.g., Ebola, and Marburg; Orthomyxoviridae, e.g., influenza; Arenaviridae, e.g., Lassa; and Retroviridae, e.g., HIV and HTLV. In specific embodiments, the viral binding polypeptide binds to a viral coat protein from HIV, Amphovirus, Marburg virus, Dengue virus, Ebola virus and SARS virus. In a related embodiment, the viral coat protein is a glycoprotein, e.g., HIV gp120, SIV gp120, Ebola GP, Cytomegalovirus gB, Hepatitis C virus E1, Hepatitis C virus E2, and Dengue virus gE. In a specific embodiment, the viral coat protein is HIV gp120.

In one embodiment, the viral binding polypeptide is a DC-SIGN polypeptide, e.g., the DC-SIGN polypeptide as set for the in SEQ ID NO:3 and encoded by the nucleic acid sequence as set forth in SEQ ID NO:4, or a biologically active fragment thereof. In a related embodiment, the DC-SIGN polypeptide is encoded by a nucleic acid that is at least 90% identical to the sequence set forth as SEQ ID NO:4.

In one embodiment, the polypeptide linker is comprised of glycine and serine amino acid residues. In a specific embodiment, the liker has the sequence (GlyGlyGlySer)₄.

In a specific embodiment, the invention provides polynucleotide encoding a polypeptide comprising a phospholipase A2, or a biologically active fragment thereof, and DC-SIGN polypeptide connected by a peptide linker. In a related embodiment, the polynucleotide has the sequence as set forth in SEQ ID NO:6.

In another aspect, the invention provides a vector comprising any one of the nucleic acid molecules of the invention as set forth herein. In a related embodiment, the vector is an expression vector.

In another aspect, the invention provides a host cell comprising an expression vector disclosed herein.

In another aspect, the invention provides a method of producing a polypeptide of the invention comprising culturing a host cell of the invention under conditions appropriate for protein expression, thereby producing the polypeptide.

In another aspect, the invention provides a pharmaceutical composition comprising an effective amount of a polypeptide of the invention and a pharmaceutically acceptable carrier.

In another aspect, the invention provides a pharmaceutical composition comprising an effective amount of a polynucleotide of the invention and a pharmaceutically acceptable carrier.

In another aspect, the invention provides a method of treating a subject having a viral infection by administering to the subject an effective amount of any one of the polypeptides of the invention, an effective amount of a polynucleotide of the invention, or a pharmaceutical composition of the invention, thereby treating a subject having a viral infection.

In one embodiment the viral infection is caused by an enveloped virus. In a related embodiment, the enveloped virus is a Herpesviridae virus, e.g., herpes and CMV; Poxyiridae virus, e.g., variola and smallpox; Hepadnaviridae virus, e.g., hepatitis B virus; Togaviridae virus, e.g., Rubella; Flaviviridae virus, e.g., hepatitis C virus and yellow fever virus; Coronaviridae virus, e.g., SARS; Paramyxoviridae virus, e.g., PIV, RSV and measles; Bunyaviridae virus, e.g., Hantavirus; Rhabdoviridae virus, e.g., VSV and rabies; Filoviridae virus, e.g., Ebola, and Marburg; Orthomyxoviridae virus, e.g., influenza; Arenaviridae virus, e.g., Lassa; or Retroviridae virus, e.g., HIV and HTLV. In specific embodiments, the enveloped virus is HIV, Hepatitis, Amphovirus, Marburg, Ebola, Herpes Simplex Virus (HSV) Type 1, Herpes Simplex Virus Type 2, Vesicular Stomatitis Virus (VSV), a Visna Virus (VV), a Measles Virus (MV), or a SARS infection.

In another aspect, the instant invention provides a method for preventing an infection in a subject by administering to the subject an effective amount of any one of the polypeptides of the invention, an effective amount of a polynucleotide of the invention, and/or a pharmaceutical composition of the invention, thereby preventing a viral infection in a subject.

In one embodiment, the viral infection is caused by an enveloped virus, e.g., HIV, Hepatitis, Amphovirus, Marburg, Ebola, Herpes Simplex Virus (HSV) Type 1, Herpes Simplex Virus Type 2, Vesicular Stomatitis Virus (VSV), Visna Virus (VV), a Measles Virus (MV), or SARS.

In another aspect, the invention provides methods of treating a subject having a viral infection by administering to the subject an effective amount of a phospholipase polypeptide, or a biologically active fragment thereof, thereby treating the subject.

In one embodiment, the phospholipase is a mammalian phospholipase, e.g., a human phospholipase. In one embodiment, the phospholipase polypeptide, or biologically active fragment thereof, is a phospholipase A polypeptide, or biologically active fragment thereof. In another embodiment, the phospholipase A polypeptide, or biologically active fragment thereof, is a phospholipase A2 polypeptide, or biologically active fragment thereof. In one exemplary embodiment, the phospholipase A2 polypeptide is a group X phospholipase A2.

In another embodiment, the viral infection is caused by an enveloped virus. In one embodiment, the viral infection is caused by an enveloped virus, e.g., HIV, Hepatitis, Amphovirus, Marburg, Ebola, Herpes Simplex Virus (HSV) Type 1, Herpes Simplex Virus Type 2, Vesicular Stomatitis Virus (VSV), Visna Virus (VV), a Measles Virus (MV), or SARS. In a specific embodiment, the virus is a Retriviridae virus, e.g., a lentivirus such as HIV or SIV.

In another aspect, the invention provides methods of treating a subject having a viral infection by administering to the subject an effective amount of a nucleic acid molecule that encodes a phospholipase polypeptide, or functional fragment thereof, or an agent that increases the expression of endogenous phospholipase in a subject, thereby treating the subject having a viral infection.

In one embodiment, the phospholipase is a mammalian phospholipase, e.g., a human phospholipase. In one embodiment, the phospholipase polypeptide, or biologically active fragment thereof, is a phospholipase A polypeptide, or biologically active fragment thereof. In another embodiment, the phospholipase A polypeptide, or biologically active fragment thereof, is a phospholipase A2 polypeptide, or biologically active fragment thereof. In one exemplary embodiment, the phospholipase A2 polypeptide is a group X phospholipase A2.

In another embodiment, the viral infection is caused by an enveloped virus. In one embodiment, the viral infection is caused by an enveloped virus, e.g., HIV, Hepatitis, Amphovirus, Marburg, Ebola, Herpes Simplex Virus (HSV) Type 1, Herpes Simplex Virus Type 2, Vesicular Stomatitis Virus (VSV), Visna Virus (VV), a Measles Virus (MV), or SARS. In a specific embodiment, the virus is a Retriviridae virus, e.g., a lentivirus such as HIV or SIV.

### DESCRIPTION OF THE DRAWINGS

FIGS. 1A-B depict exemplary molecules of the invention. FIG. 1A is a schematic representation of molecules of the invention and control molecules used in the validation of the activity of the disclosed molecules. FIG. 1A depicts a PLA2 molecule attached by a (GGGS)₄ linker to a carbohydrate recognition domain (CRD); a PLA2 molecule attached by a (GGGS)₄ linker to a mutant CRD (encoded by insert in the nucleic acid vector set forth as SEQ ID NO:7); and a PLA2 mutant molecule attached by a (GGGS)₄ linker to a CRD (encoded by insert in the nucleic acid vector set forth as SEQ ID NO:8). FIG. 1B is a Western blot showing expression of the polypeptides depicted in FIG. 1A after being cloned into a CMV/R expression vector and being transformed into 293 cells.

FIG. 2 depicts the results of a secreted phospholipase A2 assay (sPLA2). An ELISA was performed to measure sPLA2 in 10 ul of the wild-type, CRD mutant, and PLA2 mutant construct-transfected cell culture supernatants. Bee venom was used as a positive control.

FIGS. 3A-C depict the effects of the exposure of molecules of the invention on pseudo-typed lentiviral infection to MAGI-CCR5 cells for HIV, VSV-G, and Amphovirus, in A, B and C, respectively.

FIG. 4 depicts the effects of the exposure of the molecules of the invention on pseudo-typed lentilviral infection in 7860 cells for Marburg virus, Ebola virus, SARS, Ampho virus, and VSV-G, in A, B, C, D, and E, respectively.

FIG. 5 depicts the effects of PLA2-linker-CRD exposure on HIV-1 infection in A3R5 cells. p24 levels were determined by flow cytometry using an anti-p24-FITC antibody.

FIG. 6 depicts the effects of PLA2-linker-CRD exposure on HIV-1 infection in A3R5 cells. p24 levels were determined by ELISA after 3, 7 and 9 days.

FIGS. 7A-B set forth polypeptide and nucleic acid sequence of human phospholipase A2 (SEQ ID NO:1 and 2, respectively).

FIGS. 8A-B set forth polypeptide and nucleic acid sequence of DC-SIGN (SEQ ID NO:3 and 4, respectively).

FIGS. 9A-B set forth the sequence of an exemplary fusion molecule of the invention. The polypeptide and nucleic acid sequence of the phospholipase A2-linker-DC-SIGN molecule are set forth as SEQ ID NO:5 and 6, respectively.

FIGS. 10A-B sets forth a vector map and the nucleic acid sequence of the vector encoding the PLA2 molecule attached to a mutant CRD by a (GGGS)₄ linker (SEQ ID NO:7), respectively.

FIGS. 11A-B sets forth a vector map and the nucleic acid sequence of the vector encoding the PLA2 mutant molecule attached to a CRD by a (GGGS)₄ linker (SEQ ID NO:8), respectively.

FIGS. 12 A-B depict the anti-viral effect of sPLA2-X isoform is specific. (A)

The enzymatic activity of each indicated sPLA2 gene product in culture supernatant was assessed by a colorimetric assay using an sPLA2 assay kit (upper panel). Expression in supernatants was determined by Western blot analysis with anti-His antibody (lower panel). †, p<0.05; *; p<0.01 compared to control. (B) HIV-1_{IIIB} envelope-pseudotyped lentiviral vector encoding luciferase (100 μl each) was incubated with 1 ml of cell culture supernatant, made from control or the indicated sPLA2 isoform from transfected 293 cells, for 60 min at 37° C. The virus-ell culture supernatant mixture was added to MAGI-CCR5 and incubated for 16 hr. The mixture was removed, and luciferase reporter activity was evaluated 48 hrs after replacement with fresh media. The data are represented as the average +/−standard deviation from triplicates and is representative of two independent experiments.

FIGS. 13A-B depict the anti-viral effect of sPLA2-X: dependence on enzymatic activity on the virus and not target cells and specificity of inhibition. (A) sPLA2-X acts on virus rather than producer cells. The enzymatic activity of purified sPLA2-X or the inactive ΔsPLA2-X (D47K) mutant made from 293 cells was assessed by an sPLA2 assay kit (left upper panel). Protein amounts in 10 μl are shown by Western blot using anti-His antibody (left lower panel). HIV-1_{ADA} pseudovirions were incubated with sPLA2-X or inactive ΔsPLA2-X (0.3 ml) for 60 min at 37° C. and ultracentrifuged at 48,400×g for 1 hr to pellet the virus. Viral pellets were resuspended with fresh medium and incubated with the MAGI-CCR5 target cells for 17 hrs. Infectivity was assessed with a luciferase reporter 48 hrs after replacement with fresh medium (middle panel). MAGI-CCR5 target cells were incubated with sPLA2-X or the catalytically inactive ΔsPLA2-X (D47K) (0.3 ml) for 2 hours at 37° C., washed, and transduced with pseudotyped HIV-1_{ADA} virions. Cells were again washed at indicated times to remove the virions and cultured with fresh medium. Infectivity was assessed by luciferase reporter activity 3 days later (right panel). (B) sPLA2-X exerts specific anti-viral activity. Cell culture supernatant (1 ml) made from sPLA2-X (activity=33 to 78 nmol/min/ml) or ΔsPLA2-X (D47K=catalytically inactive mutant)-transfected 293 cells were incubated for 60 min at 37° C. with indicated pseudovirions. The virus supernatant mixture was added to MAGI-CCR5 (HIV-1_{ADA}, HIV-1_{IIIB}, and MoMuLV) or 786-O cells (Ebola and Ad5), incubated for 16 hrs, replaced with fresh medium, and luciferase-reporter activity was assayed 48 hrs later. The data are represented as the average ±standard deviation from triplicates.

FIGS. 14A-B depict sPLA2-X inhibits productive HIV-1 replication of CCR5- or CXCR4-tropic strains in T cells. HIV-1_{BaL} (A) or (B) HIV-1_{MN} (p24=100 ng) stocks were incubated with 53 ng of purified sPLA2-X (400 nmol/min activity; left panel) and Δ3sPLA2-X (H46N, D47E and Y50F; right panel) for 60 min at 37° C. The virus-sPLA2 mixture was incubated with the human T leukemia cell line A3R5 (a subline of CEM expressing both CCR5 and CXCR4; 1×10⁶), for 2 hours. Cells were then washed and replaced with fresh medium. HIV-1 replication was analyzed 64 h after infection by flow cytometry, staining for intracellular p24 by FITC-conjugated anti-p24 antibody. p24 positive cell percentage was subtracted from the mock infected cells.

FIGS. 15A-B depict sPLA2-X potently damages viral membranes compared to antibody-mediated complement fixation. (A) 13C6, a complement-fixing antibody, binds to Ebola pseudovirions but does not damage the viral membrane like sPLA2-X. Gradient-purified Ebola pseudovirions were incubated with a control mouse IgG or 13C6 for 30 min at 4° C. and immunoprecipitated with protein G-sepharose. Immunoprecipitate was analyzed for p24 by Western blot analysis using human anti-HIV-1 IgG (left panel). Gradient-purified Ebola pseudotyped virions were incubated with mouse IgG (67 μg/ml) or 13C6 (333 μg/ml) plus mouse complement (10%) for 90 min at 37° C. (right top panels as indicated), or 1 ml of sPLA2-X or ΔsPLA2-X (D47K) from transfected 293 cell culture supernatants for 60 min at 37° C. (right bottom panels as indicated). Density gradient was formed by centrifugation using OptiPrep and the fractions were collected. p24 Gag in each fraction is shown by Western blot analysis with anti-HIV-1 IgG. Gag released from damaged virus forms aggregates found in higher density fractions. (B) 2F5, an antibody known to fix complement, binds to HIV-1_{BaL} but does not damage the viral membrane like sPLA2-X. Purified live HIV-1_{BaL} was incubated with KZ52 (IgG1) or 2F5 (IgG1) for 30 min at 4° C. and immunoprecipitated with protein G-sepharose. Immunoprecipitate was analyzed for p24 by Western blot analysis using anti-p24 rabbit serum for the presence of 2F5 bound to HIV-1_{BaL} (left panel). HIV-1_{BaL} was incubated with 100 μg/ml of 2F5 or KZ52 monoclonal antibody with human complement (10%; right top panel as indicated), or 1 ml of culture supernatants from sPLA2-X or ΔsPLA2-X (D47K)-transfected 293 cells (right bottom panel as indicated), for 3 hrs at 37° C. Density gradient was performed by centrifugation using OptiPrep and the fractions were collected. p24 Gag in each fraction is shown by Western blot analysis using rabbit anti-p24 Gag serum. Gag released from damaged virus forms aggregates found in higher density fractions.

FIG. 16 depicts HIV-1_{BaL} transfer from dendritic cells to CD4⁺ T cells. Plasmacytoid dendritic cells (pDC), myeloid dendritic cells (mDC) and poly (I-C) treated mDCs (3×10⁴ cells) isolated from elutriated monocytes of a single donor were either mock infected (control) or infected with HIV-1_{BaL} (50 ng of p24) for 2 hrs and washed. Primary PHA-IL-2-stimulated autologous CD4⁺ T cells (1.25×10⁵ cells) were added to both mock-infected and HIV-1-infected DCs and incubated for another 72 hrs. p24 Gag in CD3⁺ cells was then analyzed by flow cytometry.

FIG. 17 depicts the effect of sPLA2-X exposure on HIV-1_{BaL} trans-infection from mDC to CD4⁺ T cells. Wild-type HIV-1_{BaL} (30 ng of p24) was added to either sPLA2-X (100 nmol/min activity) or equivalent amount (by weight) of catalytically inactive D47K mutant of sPLA2-X (ΔsPLA2-X) for 60 min before infection of poly (I:C)-treated mDCs (4×10⁴ cells each) for 2 hrs (A and B). Alternatively, viruses were directly used to infect poly (I:C) treated mDCs (C). mDCs were washed five times to remove virus and incubated with autologous CD4⁺ T cells alone (1.2×10⁵ cells each) (A) or treated with sPLA2-X (100 nmol/min activity) or equivalent amount of ΔsPLA2-X and CD4⁺ T cells (1.2×10⁵ cells each) (B and C) for 2 hrs. Cells were washed three times and cultured for additional 72 hrs. p24 Gag in CD3⁺ cells was then analyzed by flow cytometry. % transfer was shown in the right panel (Δ=ΔsPLA2-X, and WT=sPLA2-X).

FIG. 18 depicts the comparison of the effects of sPLA2-X and neutralizing antibodies on HIV-1_{BaL} trans-infection from mDCs to CD4⁺ T cells. Poly (I:C)-treated mDCs were infected with HIV-1_{BaL} for 2 hrs, washed five times, and incubated with human IgG (hIgG), B12, 2F5 (each 50 μg/ml), sPLA2-X (100 nmol/min activity) or equivalent amount of catalytically inactive D47K mutant of sPLA2-X (ΔsPLA2-X) and primary PHA-IL-2 stimulated autologous CD4⁺ T cells for 2 hrs. Cells were washed 3 times and cultured for another 72 hrs. p24 Gag in CD3⁺ cells was assayed by flow cytometry. % transfer was defined as the number of p24-Gag positive cells compared to the number in control wells (no antibody or no sPLA2-X) during transfer.

FIG. 19 sets forth the amino acid and nucleic acid sequence of human group 10× phospholipase A2 (SEQ ID NOs:25 and 26, respectively). The coding region of SEQ ID NO:25 encompasses nucleic acid residues 441-938.

### DETAILED DESCRIPTION OF THE INVENTION

The instant invention is based, at least in part, on the discovery that phospholipase molecules are effective broad spectrum antiviral agents. Moreover, fusion molecules comprising an enzyme, for example a phospholipase, attached to a viral binding polypeptide, e.g., a polypeptide that binds to a glycoprotein or a carbohydrate such as a lectin, and optionally including a linker, are effective broad spectrum antiviral agents. In some embodiments, the invention provides polypeptides having a phospholipase covalently attached to one end of a linker and a viral targeting polypeptide covalently attached to the other end of the linker. The molecules of the invention are useful in the treatment of viral infection caused by enveloped viruses, e.g., HIV, hepatitis, and SARS. Accordingly, the instant invention further provides pharmaceutical compositions and methods of treating viral infection using the molecules of the invention.

Molecules of the Invention

The present invention provides polypeptide and nucleic acid fusion molecules, e.g., molecules comprising an enzyme, e.g., a phospholipase, attached to a viral binding polypeptide, e.g., a carbohydrate recognition polypeptide, or biologically active fragment thereof. The phospholipase and the polypeptide that bind to an enveloped virus are optionally connected by a linker, e.g., a peptide or non-peptide linker. The invention provides molecules having the phospholipase C-terminal to the viral binding polypeptide and N-terminal to the viral binding peptide. For example, the molecules of the invention can be designed as follows: P-V, V-P, P-L-V, V-L-P, wherein V represents the viral binding polypeptide, P represents the phospholipase, and L represents the linker.

The term “viral binding polypeptide” is intended to mean a polypeptide, or fragment thereof, that recognizes and binds to a virus. In certain embodiments the viral binding polypeptide, or fragment thereof, binds to a protein on the viral coat, e.g., a glycoprotein such as gp120 on HIV. In other embodiments, the viral binding polypeptide, or fragment thereof, binds to a carbohydrate, e.g., a lectin, on a virus. In a specific embodiment the viral binding polypeptide is DC-SIGN.

Exemplary phospholipases include mammalian phospholipases, e.g., human, bovine, or murine phospholipases. In specific embodiments, the phospholipase is phospholipase A2 such as the human phospholipase A2 (the amino acid and nucleic acid sequence of which are set forth as SEQ ID NO:1 and 2, respectively). Moreover, one of skill in the art will understand that the phospholipase of the invention may be a biologically active fragment of a phospholipase, e.g., a portion of a phospholipase polypeptide that retains enzymatic, e.g., phospholipase, activity. Alternatively, the phospholipase used in the methods of the invention can be chosen to increase the specificity and efficacy of the molecules of the invention. For example, a phospholipase selected from the group consisting of a phospholipase A, a phospholipase B, a phospholipase C, and a phospholipase D. Moreover, mammalian derived phospholipases are preferred, however, non-mammalian sources may be used to alter the specificity and/or efficacy of the molecules of the invention.

In another embodiment, the phospholipase of the invention is a group X phospholipase A2, (the amino acid and nucleic acid sequence of which are set forth as SEQ ID NO:25 and 26, respectively) (GenBank Accession Nos.: NM_{—}003561 and NP_{—}003552, respectively). In related embodiments, the group X phospholipase A2 can be a biologically active fragment of the full length polypeptide. For example, the biologically active fragment can be a fragment that maintains the phospholipase activity, e.g., residues 43 to 157 of SEQ ID NO:26.

One of skill in the art can identify other phospholipases and understands that homologues and orthologues of these molecules are useful in the compositions and methods of the instant invention. Moreover, variants of phospholipases are useful in the methods and compositions of the invention. Phospholipases are described in, for example, Chakraborti, S. (2003) Cell Signal 15:637-65, Fukami, K. (2002) J. Biochem (Tokyo) 131:29309, Dessen, A. (2000) Biochim. Biophys. Acta. 1488:4047, Rebecchi, M. J. et al. (2000) Physiol. Rev. 80:1291-335, Ktistakis, N. T. et al. (1999) Biochem. Soc. Trans. 27:634-7, and Maury, E. et al. (2002) Biochem. Biophys. Res. Commun. 12:362-9.

Phospholipase A2s are a family of proteins that have conserved enzymatic domains that are approximately 120 amino-acid in length, have four to seven disulfide bonds, and release fatty acids from the second carbon group of glycerol. Phospholipase A2s bind a calcium ion which is required for activity. The side chains of two conserved residues, a histidine and an aspartic acid, participate in a catalytic network which allows for the catalytic activity of the enzyme. The conserved motifs comprising the histidine and aspartic acid are C-C-{P}-x-H-{LGY}-x-C and [LIVMA]-C-{LIVMFYWPCST}-C-D-{GS}-x(3)-{QS}-C, respectively (see, Prosite PDOC00109, Gomez, F., et al. (1989) Eur. J. Biochem. 186:23-33 and Davidson, F. F., et al. (1990) J. Mol. Evol. 31:228-238). Residues that are not involved formation of the disulfide bonds, binding of the calcium ion, in the conserved motifs comprising the histidine or aspartic acid moieties described above, or in the catalytic activity of the enzyme are more likely to be substituted or deleted without altering the activity of the enzyme.

Variants of the polypeptides used in the methods of the instant invention may have one or more conservative amino acid substitutions. Conservative amino acid substitutions are detailed in Creighton, Proteins (1984) and are set forth below. Residues on the same line are considered to be conservative substitutions for each other.

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

The term “biologically active fragment thereof” refers to peptides and polypeptides that are derived from a phospholipase or viral binding polypeptide and that retain the same or similar activity of a phospholipase or a viral binding polypeptide, e.g., a polypeptide that retains the enzymatic activity of a phospholipase or the binding activity of a viral binding polypeptide, e.g., the ability to bind to a glycoprotein and/or a carbohydrate.

The phospholipase is attached to a viral binding polypeptide. This polypeptide, (sometimes referred to herein as a carbohydrate recognition domain), is any polypeptide that has the ability to bind to an enveloped virus, and more specifically, has the ability to bind to a protein or carbohydrate presented by an enveloped virus. In at least one specific embodiment, the viral binding polypeptide binds to a lectin, e.g., DC-SIGN. Exemplary viral binding polypeptides include those that recognize glycoproteins expressed by a virus, e.g., the gp120 protein from HIV, the gp120 protein from SIV, the GP protein from Ebola, the gB protein from cytomegalovirus, the E1 protein from Hepatitis C, the E2 protein from Hepatitis C or the gE protein from Dengue virus. Moreover, specific exemplary polypeptides include lectin binding proteins and fragments thereof such as the carbohydrate recognition domain of DC-SIGN (the amino acid and nucleic acid sequence of which are set forth as SEQ ID NO:3 and 4, respectively).

One of skill in the art will understand that molecules that share one or more functional activities with the molecules identified above, but have differences in amino acid or nucleic acid sequence would be useful in the compositions and methods of the invention. For example, in a preferred embodiment, a polypeptide or biologically active fragment thereof has at least about 60%, 70%, 80%, 90%, 95%, 96%, 97%, 98%, 99%, or 100% identity with the polypeptide set forth as SEQ ID NO:1, 3 or 26, or a fragment thereof. Accordingly, variants of full length human phospholipase A2 polypeptides that are 60%, 70%, 80%, 90%, 95%, 96%, 97%, 98%, 99% identical to human phospholipase A2 (SEQ ID NO:1) would have 99, 112, 132, 149, 157, 158, 160, 162 and 163 identical residues, respectively. Further, variants of the CRD of DC-SIGN that are 60%, 70%, 80%, 90%, 95%, 96%, 97%, 98%, 99% identical the CRD of DC-SIGN (SEQ ID NO:3) would have 93, 116, 124, 140, 147, 150, 152, and 154 identical residues, respectively.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970, J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

The percent identity between two amino acid or nucleotide sequences can be determined using the algorithm of Meyers et al. (1989, CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences that one of skill in the art could use to make the molecules of the invention. Such searches can be performed using the NBLAST and XBLAST programs (version 2.0) of Altschul, et al. (1990, J. Mol. Biol. 215:403-410). BLAST nucleotide searches can be performed with the NBLAST program, score=100, wordlength=12 to obtain nucleotide sequences homologous to the nucleic acid molecules used in the methods and compositions of the invention. BLAST protein searches can be performed with the XBLAST program, score=50, wordlength=3 to obtain amino acid sequences homologous to the molecules used in the methods and compositions of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used.

One of skill in the art understands that two or more DNA sequences that differ from each other may encode the identical, or nearly identical, protein molecules due to the degeneracy of the genetic code. See Table 1.

Table 1 depicts the degeneracy of the genetic code.

TABLE 1

| 1^{st} position | 2^{nd} position | | | | 3^{rd} position |
|---|---|---|---|---|---|
| (5′ end) | U(T) | C | A | G | (3′ end) |
| U(T) | Phe | Ser | Tyr | Cys | U(T) |
| | Phe | Ser | Tyr | Cys | C |
| | Leu | Ser | STOP | STOP | A |
| | Leu | Ser | STOP | Trp | G |
| C | Leu | Pro | His | Arg | U(T) |
| | Leu | Pro | His | Arg | C |
| | Leu | Pro | Gln | Arg | A |
| | Leu | Pro | Gln | Arg | G |
| A | Ile | Thr | Asn | Ser | U(T) |
| | Ile | Thr | Asn | Ser | C |
| | Ile | Thr | Lys | Arg | A |
| | Met | Thr | Lys | Arg | G |
| G | Val | Ala | Asp | Gly | U(T) |
| | Val | Ala | Asp | Gly | C |
| | Val | Ala | Glu | Gly | A |
| | Val | Ala | Glu | Gly | G |

The molecules of the invention optionally contain a linker, e.g., a polypeptide linker. In certain embodiments the linker is comprised of amino acids that allow for flexibility of the linker. In certain embodiments, the polypeptide linker consists of from about 4 to about 40 amino acid residues. In specific embodiments, the linker is comprised of glycine and serine residues. In a specific embodiment, the linker has the sequence (GlyGlyGlySer)₄. Alternatively, the polypeptides of the invention may contain a non-peptide linker, e.g., a polyethyleneglycol (PEG)linker or a alkyl linkers such as —NH—(CH₂), —C(O)—, wherein s=2-20.

The molecules of the invention may be assembled post-translationally, i.e., the phospholipase and the carbohydrate recognition domain can be covalently linked after being synthesized or expressed separately. Alternatively, a phospholipase, or biologically active fragment thereof, a viral binding polypeptide, and optionally, a linker can be expressed as a single transcript in a recombinant host cell or organism as described herein

Another aspect of the invention pertains to vectors, preferably expression vectors, containing a nucleic acid molecule encoding the fusion molecules, or components thereof, of the invention as described above. As used herein, the term “vector” refers to a nucleic acid molecule capable of transporting another nucleic acid molecule to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., a non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule of the invention in a form suitable for expression of the protein molecule in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of the polypeptides of the invention in prokaryotic or eukaryotic cells. For example, the polypeptides can be expressed in bacterial cells such as E. coli, insect cells (using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

A specific vector that can be used to express the polypeptides of the invention is a CMV/R expression vector such as those described in U.S. Ser. No. 10/997,120, filed Nov. 24, 2004 and PCT/US02/30251, filed Sep. 24, 2002.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:3140), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (M7 gnl). This viral polymerase is supplied by host strains BL21(DE3) or HMS174(DE3) from a resident prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari, et al., (1987) EMBO J 6:229-234), pMFa (Kudjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, the polypeptides can be expressed in insect cells using baculovirus expression vectors. Baculovirus vectors available for expression of proteins in cultured insect cells (e.g., Sf9 cells) include the pAc series (Smith et al. (1983) Mol. Cell. Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banedji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Another aspect of the invention pertains to host cells into which a nucleic acid molecule encoding a fusion polypeptide of the invention is introduced within a recombinant expression vector or a nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a fusion polypeptide of the invention can be expressed in bacterial cells such as E. coli, insect cells, yeast or mammalian cells (such as Chinese hamster ovary cells (CHO), COS cells, or human embryonic kidney (HEK) 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, methotrexate, kanamycin, ampicillin, chloramphenicol, and tetracycline. Nucleic acid encoding a selectable marker can be introduced into a host cell on the same vector as that encoding the polypeptide of the invention or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) the polypeptides of the invention. Accordingly, the invention further provides methods for producing polypeptides using the host cells of the invention. In one embodiment, the method comprises culturing the host cell of the invention (into which a recombinant expression vector encoding a polypeptide of the invention has been introduced) in a suitable medium such that a polypeptides of the invention is produced. In another embodiment, the method further comprises isolating the polypeptide from the medium or the host cell.

The host cells of the invention can also be used to produce non-human transgenic animals. For example, in one embodiment, a host cell of the invention is a fertilized oocyte or an embryonic stem cell into which coding sequences have been introduced. Such host cells can then be used to create non-human transgenic animals in which exogenous sequences have been introduced into their genome or homologous recombinant animals in which endogenous sequences have been altered. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like.

Methods of Making the Molecules of the Invention

As described above, molecules of the invention may be made recombinantly using the nucleic acid molecules, vectors, host cells and recombinant organisms described above.

Alternatively, the phospholipase, or fragment thereof, and/or the viral binding polypeptide, or fragment thereof, can be made synthetically or isolated from a natural source and linked together using methods and techniques well known to one of skill in the art.

Further, to increase the stability or half life of the molecules of the invention, the polypeptides may be made, e.g., synthetically or recombinantly, to include one or more peptide analogs or mimetics. Exemplary peptides can be synthesized to include D-isomers of the naturally occurring amino acid residues or amino acid analogs to increase the half life of the molecule when administered to a subject.

Pharmaceutical Compositions

The nucleic acid and polypeptide molecules (also referred to herein as “active compounds”) of the invention can be incorporated into pharmaceutical compositions. Such compositions typically include the nucleic acid molecule or protein, and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions of the instant invention may also include one or more other active compounds. Alternatively, the pharmaceutical compositions of the invention may be administered with one or more other active compounds. Other active compounds that can be administered with the pharmaceutical compounds of the invention, or formulated into the pharmaceutical compositions of the invention, include, for example, other antiviral compounds.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Preferred pharmaceutical compositions of the invention are those that allow for local delivery of the active ingredient, e.g., delivery directly to the location of a tumor. Although systemic administration is useful in certain embodiments, local administration is preferred in most embodiments.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, kit or dispenser together with instructions, e.g., written instructions, for administration, particularly such instructions for use of the active agent to treat against a disorder or disease as disclosed herein, including an viral infection. The container, pack, kit or dispenser may also contain, for example, a fusion molecule, a nucleic acid sequence encoding a fusion molecule, or a fusion molecule expressing cell.

Therapeutic and Prophylactic Methods

The present invention provides for both prophylactic and therapeutic methods of treating a subject at risk of, or susceptible to, a viral infection. Treatment is defined as the application or administration of a therapeutic agent to a patient, or application or administration of a therapeutic agent to an isolated tissue or cell line from a patient, who has a viral infection, a symptom of a viral infection or a predisposition toward a viral infection, with the purpose to cure, heal, alleviate, relieve, alter, remedy, ameliorate, improve or affect the viral infection, the symptoms of the viral infection or the predisposition toward the viral infection.

The therapeutic methods of the invention involve the administration of the polypeptide and/or nucleic acid molecules of the invention as described herein.

In one aspect, the invention provides a method for preventing a viral infection in a subject associated with an enveloped virus by administering to the subject a polypeptide or nucleic acid molecule of the invention as described herein.

As used herein “viral infection” is intended to mean an infection of a subject by a virus, e.g., an enveloped virus such as those belonging to Herpesviridae, e.g., herpes and CMV; Poxyiridae, e.g., variola and smallpox; Hepadnaviridae, e.g., hepatitis B virus; Togaviridae, e.g., Rubella; Flaviviridae, e.g., hepatitis C virus and yellow fever virus; Coronaviridae; Paramyxoviridae, e.g., PIV, RSV and measles; Bunyaviridae, e.g., Hantavirus; Rhabdoviridae, e.g., VSV and rabies; Filoviridae, e.g., Ebola, and Marburg; Orthomyxoviridae, e.g., influenza; Arenaviridae, e.g., Lassa; and Retroviridae, e.g., HIV and HTLV. In specific embodiments, the virus is HPV, hepatitis viruses A, B, C, D and E, SARS, ebola, SIV, cytomegalovirus, Dengue, Marburg, VSV, or HIV.

The invention provides therapeutic methods and compositions for the prevention and treatment of viral infection. In particular, the invention provides methods and compositions for the prevention and treatment of viral infection in subjects.

In one embodiment, the present invention contemplates a method of treatment, comprising: a) providing, i.e., administering: i) a mammalian patient particularly human who has, or is at risk of developing, a viral infection, ii) one or more fusion molecules of the invention as described herein.

The term “at risk for developing” is herein defined as individuals an increased probability of contracting an infection due to exposure or other health factors.

The present invention is also not limited by the degree of benefit achieved by the administration of the fusion molecule. For example, the present invention is not limited to circumstances where all symptoms are eliminated. In one embodiment, administering a fusion molecule reduces the number or severity of symptoms of a viral infection. In another embodiment, administering of a fusion molecule may delay the onset of symptoms of a viral infection.

Typical subjects for treatment in accordance with the individuals include mammals, such as primates, preferably humans. Cells treated in accordance with the invention also preferably are mammalian, particularly primate, especially human. As discussed above, a subject or cells are suitably identified as in needed of treatment, and the identified cells or subject are then selected for treatment and administered one or more of fusion molecules of the invention.

The invention further provides for methods of treating an individual having a viral infection by administering to the individual a polypeptide of the invention and one or more additional anti-viral compositions.

The treatment methods and compositions of the invention also will be useful for treatment of mammals other than humans, including for veterinary applications such as to treat horses and livestock e.g. cattle, sheep, cows, goats, swine and the like, and pets such as dogs and cats.

To treat an infection in a subject, one could increase the endogenous phospholipase expression using transcriptional activators or by delivering gene expression constructs through viral or non-viral gene-transfer vectors. Alternatively, phospholipase polypeptides, or active fragments thereof, can be synthesized in vitro, purified and delivered as a medicine to sites or tissues where it will likely be therapeutic. Thirdly, small molecule or other therapeutic compounds can be administered to boost the enzymatic activity of existing phospholipase.

In some embodiments, to modulate phospholipase expression or activity (e.g., for therapeutic purposes), a cell is contacted with a phospholipase nucleic acid or polypeptide (or active fragment thereof), or an agent that modulates one or more of the activities of phospholipase polypeptide activity associated with the cell. An agent that modulates phospholipase polypeptide activity can be, e.g., an agent as described herein, such as a nucleic acid or a polypeptide, a naturally-occurring binding partner of a phospholipase polypeptide (e.g., a phospholipase substrate), a phospholipase antibody, a phospholipase agonist, a peptidomimetic of a phospholipase agonist, or other small molecule. The agent can be synthetic, or naturally-occurring. The cell can be an isolated cell, e.g., a cell removed from a subject or a cultured cell, or can be a cell in situ in a subject.

A phospholipase enhancer agent can, in some embodiments, stimulate one or more phospholipase activities. Examples of such stimulatory agents include active phospholipase polypeptide or an active fragment thereof, and a nucleic acid molecule encoding a phospholipase polypeptide or active fragment thereof. In another embodiment, the agent inhibits one or more phospholipase activities. These modulatory methods can be performed in vitro (e.g., by culturing a cell with the agent) or, alternatively, in vivo (e.g., by administering the agent to a subject). Thus, an individual afflicted with a condition characterized by aberrant (i.e., decreased) expression or activity of a phospholipase polypeptide or nucleic acid molecule can be treated using a phospholipase agent. The method of treatment can involve administering an agent (e.g., an agent identified by a screening assay described herein), or combination of agents that modulates (e.g., up regulates) phospholipase expression or activity. Thus, in some embodiments, the method involves administering a phospholipase polypeptide or nucleic acid molecule as therapy to compensate for reduced phospholipase expression or activity.

Stimulation of phospholipase activity or expression is desirable in situations in which phospholipase is detrimentally downregulated and/or in which increased phospholipase activity is likely to have a beneficial effect.

As defined herein, a therapeutically effective amount of a phospholipase nucleic acid or polypeptide composition is a dosage effective to treat or prevent a particular condition for which it is administered. The dose will depend on the composition selected, i.e., a polypeptide or nucleic acid. The compositions can be administered from one or more times per day to one or more times per week; including once every other day. The skilled artisan will appreciate that certain factors may influence the dosage and timing required to effectively treat a subject, including but not limited to the severity of the condition, previous treatments, the general health and/or age of the subject, and other conditions present. Moreover, treatment of a subject with a therapeutically effective amount of the therapeutic phospholipase compositions of the invention can include a single treatment or a series of treatments, as well as multiple (i.e., recurring) series of treatments.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Construction of Antiviral Molecules of the Invention

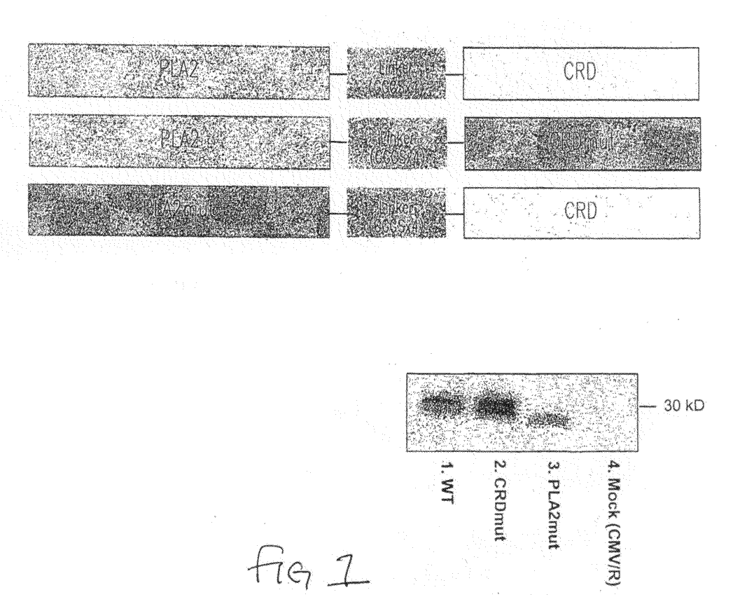

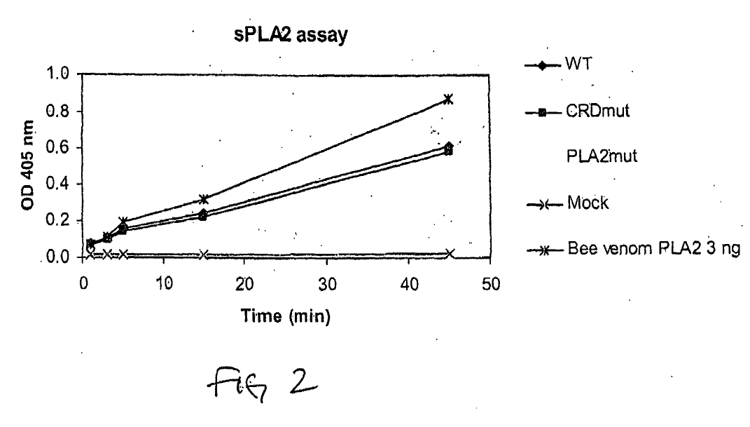

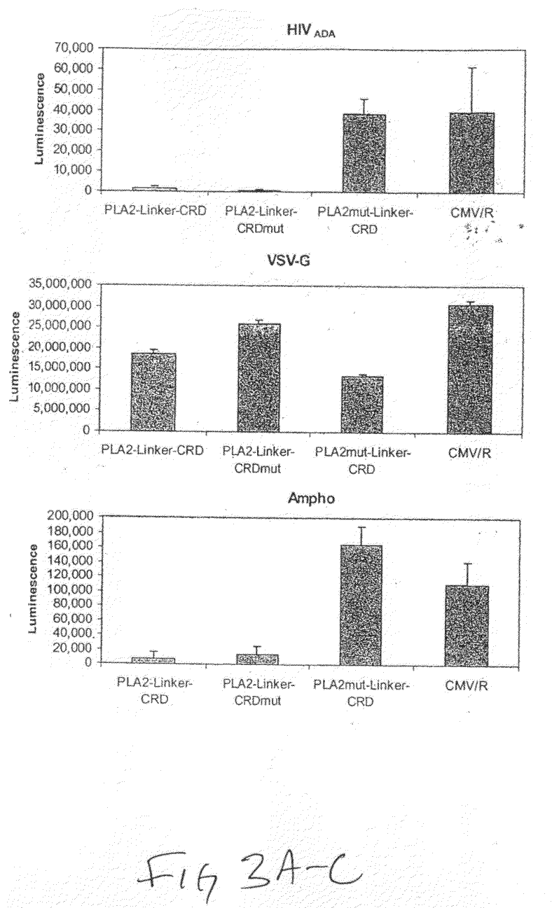

Human group X secreted phospholipase A2 (NM_{—}003561) (SEQ ID NO: 2) was PCR amplified from corresponding PLA2 cDNA clones obtained from Openbiosytems. Linker (4×GGGS) sequence was added to the carboxy-terminal of the sPLA2 group X gene using 3′primer. Carbohydrate recognition domain (CRD) of DC-SIGN (SEQ ID NO:4) was also PCR amplified from pLZR5 DCSIGN-CITE-GFP clone 2 (Nabel's lab plasmid VRC #7900) using appropriate primers. Phospholipase A2-Linker-CRD was constructed by using overlapping extension PCR of equal amounts of each PCR product. See FIG. 1.

### Example 2

### Expression of Antiviral Molecules of the Invention

Human embryonic kidney (HEK) 293 cells (5×10⁶ cells) were seeded on 100 mm-plates one day before transfection. HEK 293 cells were transfected with 10 μg of DNA using calcium phosphate (Promega).

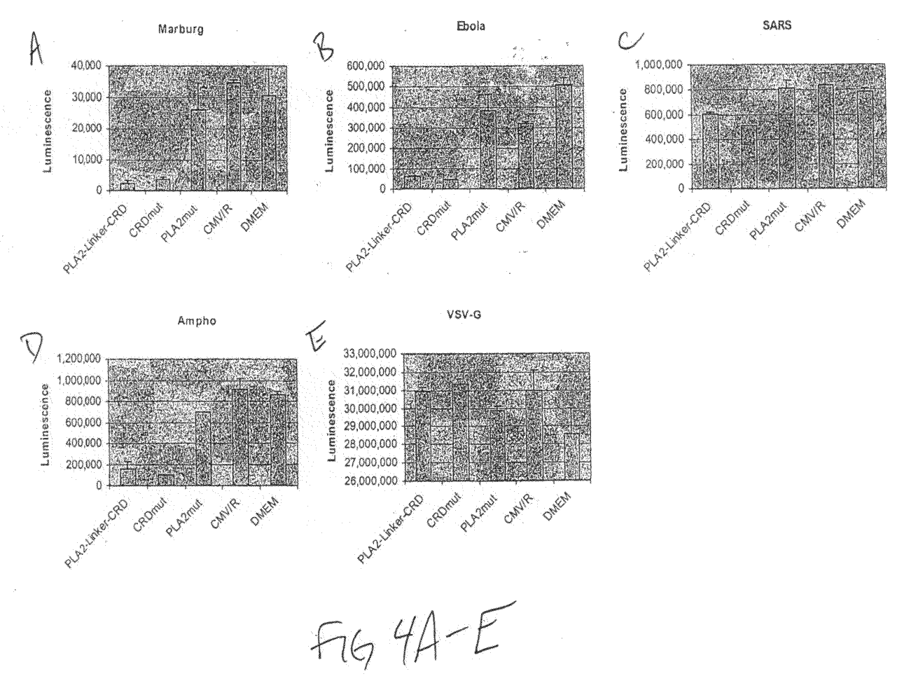

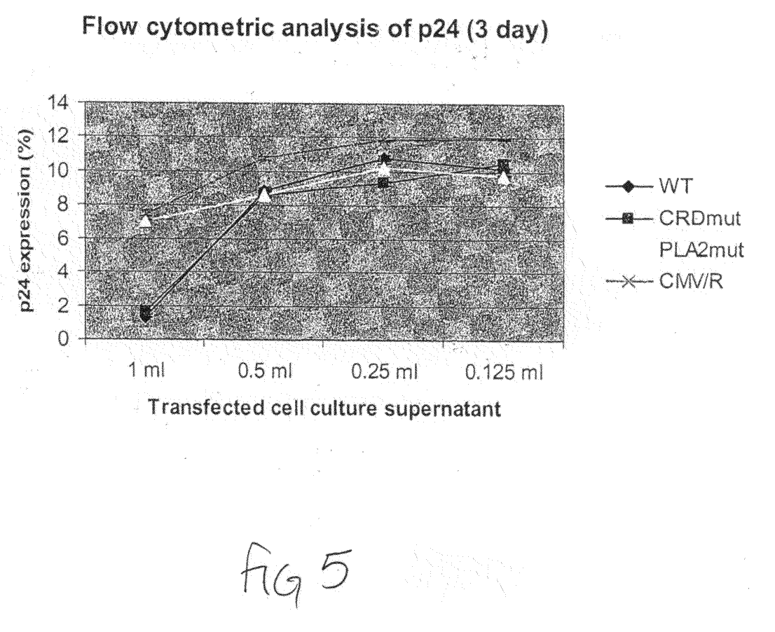

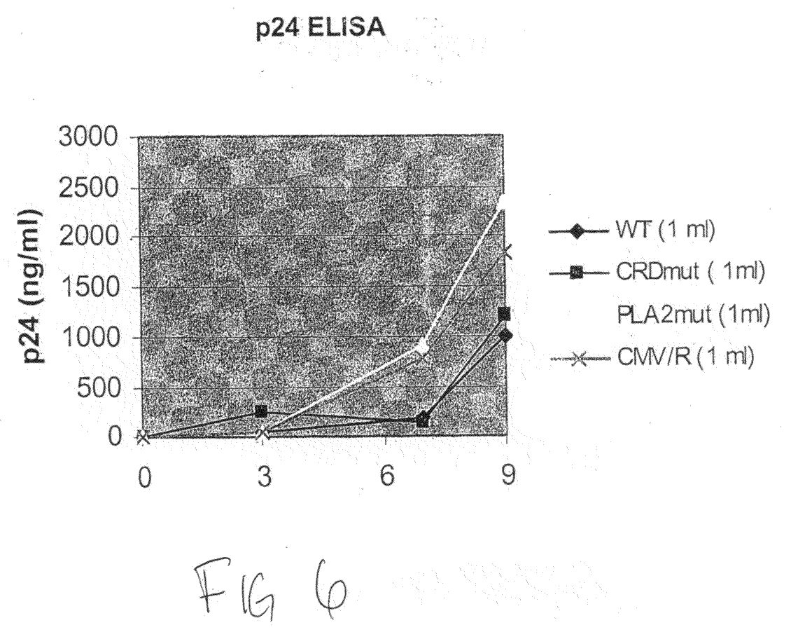

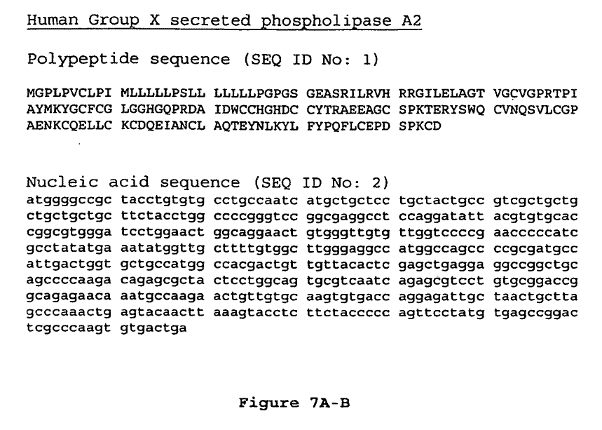

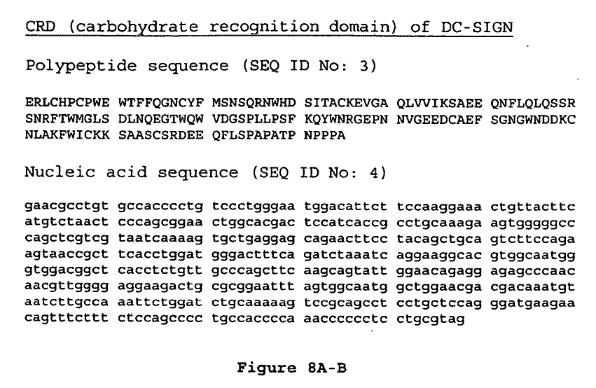

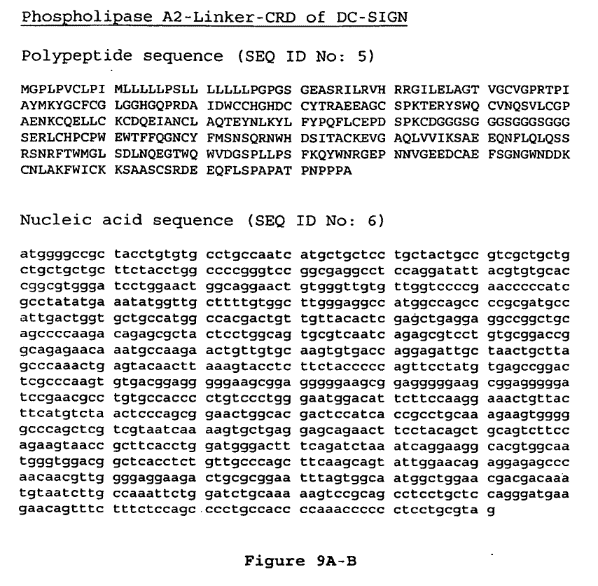

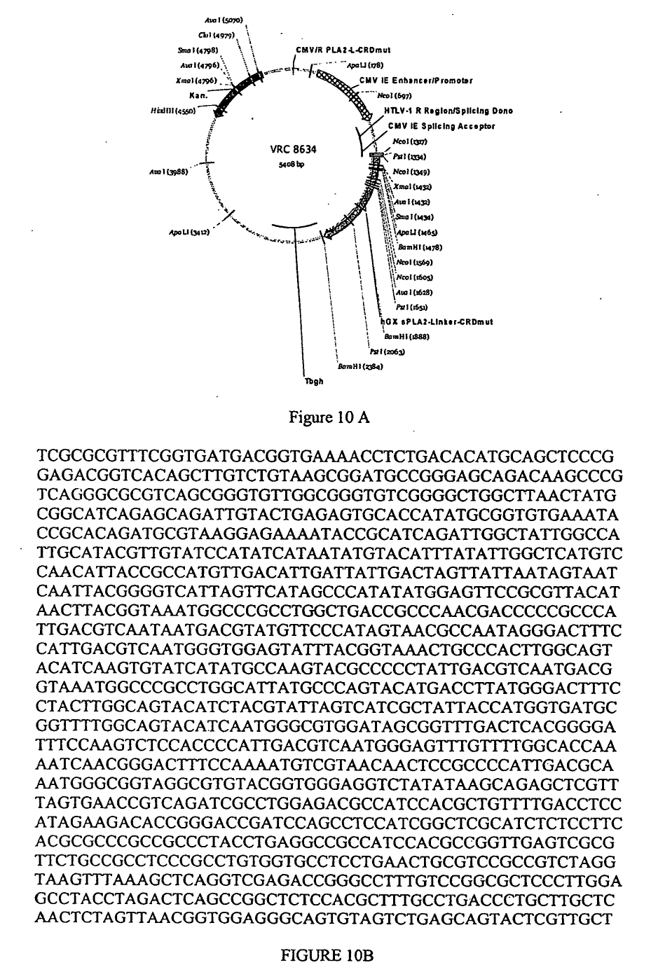

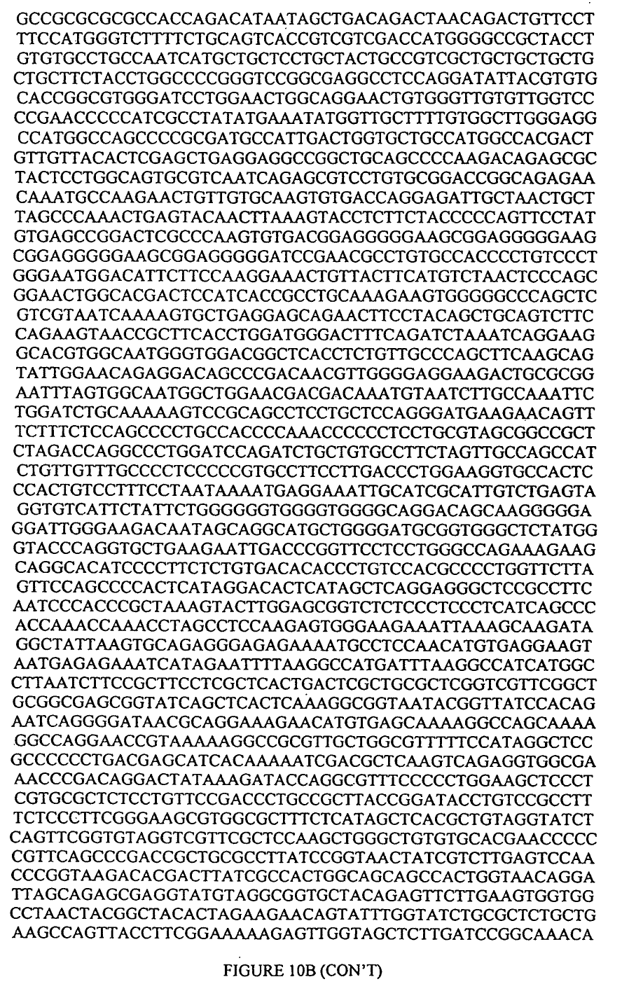

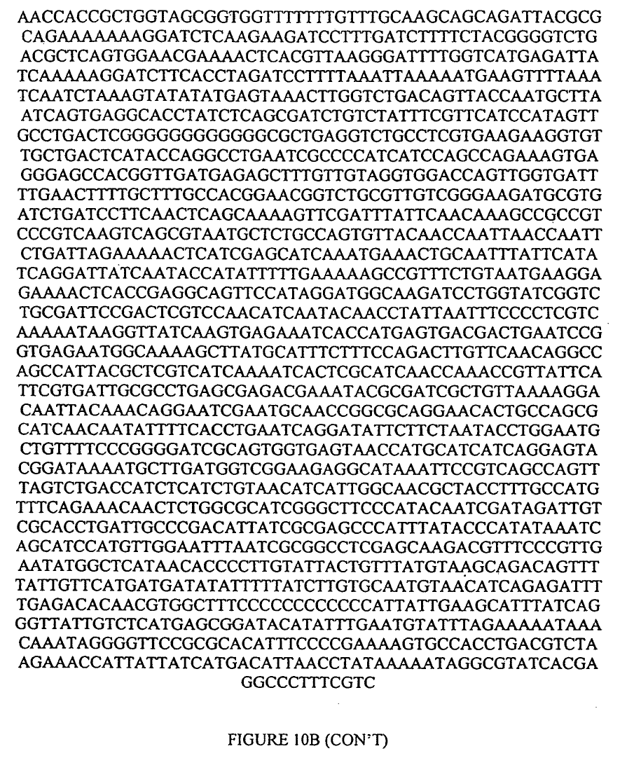

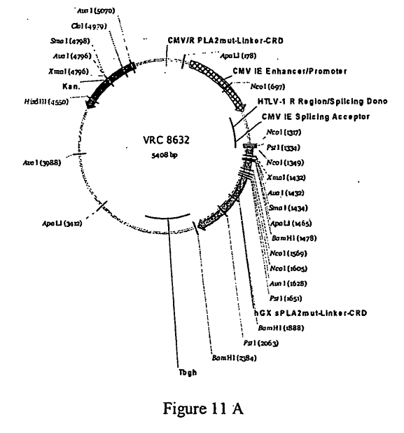

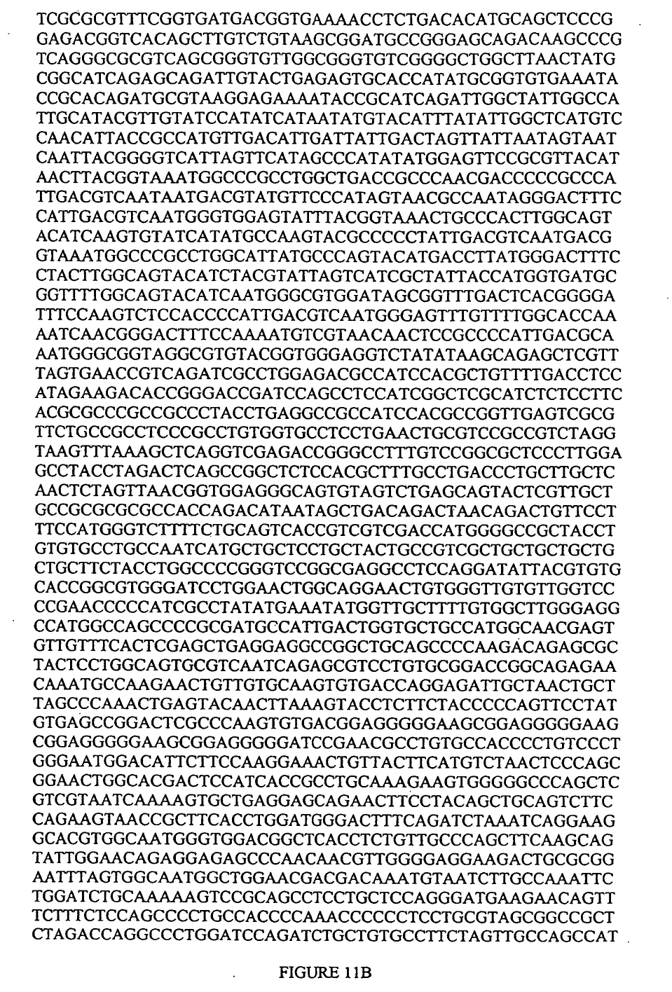

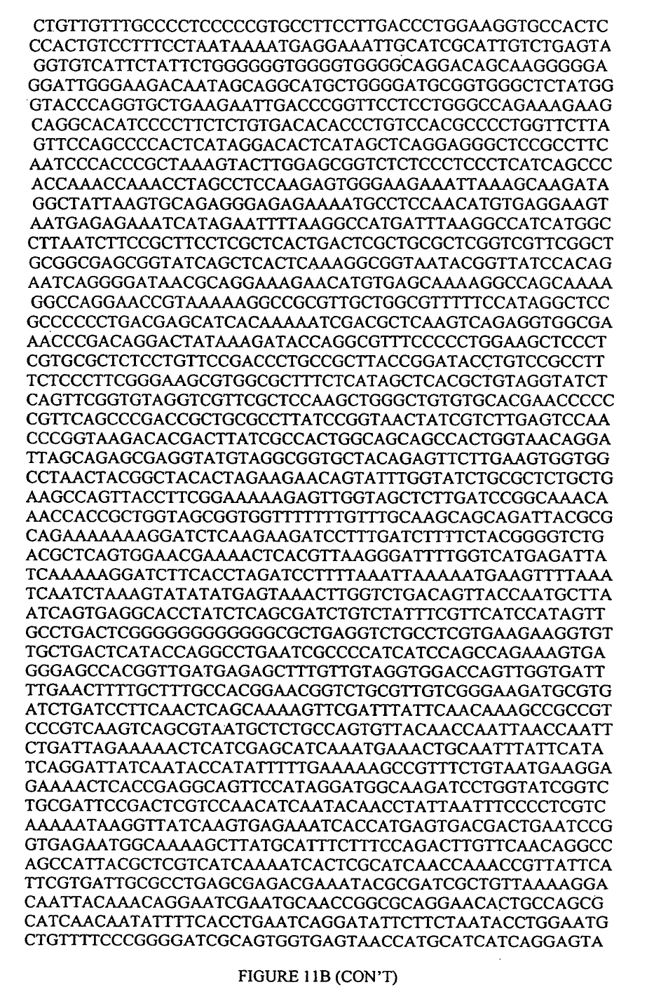

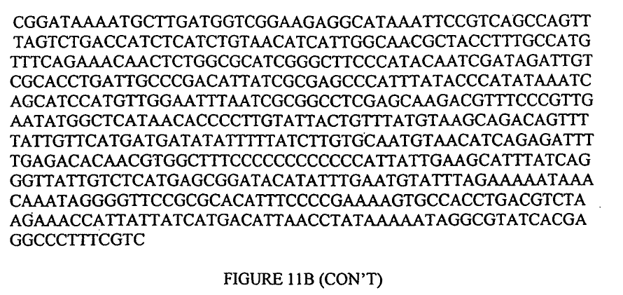

Cell culture supernatants were harvested 2 days after transfection and stored at −80° C. Expression of recombinant protein was confirmed by western blot. Briefly, cell culture supernatants were resolved by SDS-PAGE and transferred to a PVDF membrane (Bio-Rad). The membrane was incubated with rabbit polyclonal anti-DC-SIGN antibody (Oncogene) for 1 hour at room temperature in blocking buffer (Tris-buffered saline, 3% skim milk, 0.5% Triton X-100), followed by washing. The blot was further incubated in blocking buffer with horseradish peroxidase-conjugated goat anti-rabbit IgG (Santa Cruz) for 30 min and then washed. Detection was performed with the ECL reagent (Amersham). See FIG. 2.

### Example 3

### Inhibition of Pseudo-Virus Infection

Virus Preparation:

Ebola, HIV_{ADA}, HIV_{IIIB}, Marburg, and VSV envelope lentiviruses expressing luciferase were prepared by transient co-transfection of 293T cells with calcium phosphate (Promega). Briefly, the packaging vector pCMVAR8.2, pHR′CMV-Luc and the envelope expressing vector pVR1012-GP(Z), pSVIII-ADA, pRSV-IIIB, pCMV/R-Angola GP or pVSV-G supernatants were harvested 72 hours after transfection, filtered with 0.45-μm-pore-size syringe filter, and stored at −80° C.

Infection of Cells with Pseudoviruses and Luciferase Assay:

A total 30,000 cells were plated into each well of a 48-well dish the day before infection; MAGI-CCR5 was used for HIV_{ADA} and HIV_{IIIB}, and 786-0 cells were used for Ebola, Marburg, and VSV. Pseudoviral supernatant (50 to 100 μl) was incubated with cell culture supernatant from the groups indicated, for 1 hour at 37° C. then added to the target cells. Cells were replenished with fresh medium at 16 to 18 hours postinfection. After 48 hours, cells were lysed in cell lysis buffer (Promega) 80 μl in the plate and 20 μl of cell lysate was used in luciferase assay with luciferase assay reagent (Promega) according to manufacturer's recommendations. See FIG. 3.

### Example 4

### Inhibition of Live Virus Infection

Live wild-type HIV_{ADA} stocks were incubated with phospholipase A2-Linker-CRD or the mutant supernatant (1 ml) for 60 min at 37° C., added to 1×10⁵ cells of A3R5 for 60 min. Cells were then washed to remove virus, and replaced with fresh medium. HIV-1 replication was analysed 72 h after infection by flow cytometry, staining for intracellular p24 by FITC-conjugated anti-p24 antibody (KC-57 FITC; Beckman Coulter). See FIG. 4.

### Example 5

### Inhibition of Retrovirus Infection by Phospholipase A2-X

### Materials and Methods

Cell lines. The 786-O (human kidney adenocarcinoma) cell line was purchased from the American Type Culture Collection. The HeLa-derived cell line MAGI-CCR5 (a subline of HeLa expressing CCR5) was obtained from the NIH AIDS Research and Reference Reagent Program. Human T-cell leukemia cell line A3R5 (a subline of A3.01 expressing both CCR5 and CXCR4) was a gift from Dr. Jerome Kim of the Walter Reed Army Institute of Research. 293T cells were kindly provided by John Mascola. Cells were cultured with Dulbecco's modified Eagle's medium or RPMI 1640 (Invitrogen) containing 10% fetal bovine serum (Sigma) and 100 μg of penicillin-streptomycin/ml.

Construction of expression plasmids. Human sPLA2s (PLA2 group IIA: NM_{—}000300; PLA2 group IID: NM_{—}012400; PLA2 group III: NM_{—}015715; PLA2 group V: NM_{—}000929; PLA2 group VII: NM_{—}005084; PLA2 group X: NM_{—}003561; PLA2 group XIIA: NM_{—}03081) were PCR amplified from corresponding PLA2 cDNA clones obtained from Invitrogen or Openbiosytems, and subcloned into the mammalian expression vector CMV/R-mcs (Journal of Virology, June 2004, p. 5642-5650, Vol. 78, No. 11). Linker (4×GGGS) and 6×His tag were added to the carboxy-terminal of the sPLA2 group X gene and a carboxy-terminal 6×His tag alone was added to the other genes. Point mutants were constructed by using overlap extension PCR or QuickChange site-directed mutagenesis kit (Stratagene) according to the manufacturer's protocol. All plasmids were sequenced to verify the coding regions. The primer sequences for amplification of each sPLA2 isoforms are:

(SEQ ID NO:7)

IIA 5′: ACCGTTAGCGGCCGCCACCATGAAGACCCTCCTA
CTGTTGGCAGTGATCATGA

(SEQ ID NO:8)

IIA 3′: TGCCAGTTCTAGATCAATGATGATGATGATG
ATGGCAACGAGGGGTGCTCCCTCTGCAGTGTTTATTG

(SEQ ID NO:9)

IID 5′: ACCGTTAGCGGCCGCCACCATGGAACTTGCACTGCTGTGT
GGGCTGGTGGTGATGGCTGGTG

(SEQ ID NO:10)

IID 3′: TGCCAGTTCTAGATCAATGATGATGATGATGATGGCA
CCCAGGGGTCTGCCCCCGGCAGTGGGGCC

(SEQ ID NO:11)

III 5′: ACCGTTAGCGGCCGCCACCATGGGGGTTCAGGCAGGGCTG
TTTGGGATGCTGGG

(SEQ ID NO:12)

III 3′: TGCCAGTTCTAGATCAATGATGATGATGATGATGCTGGC
TCCAGGACTTCTGCTGCCTGT

(SEQ ID NO:13)

V 5′: ACCGTTAGCGGCCGCCACCATGAAAGGCCTCCTCCCACTGGC
TTGGTTCCTGGC

(SEQ ID NO:14)

V 3′: TGCCAGTTCTAGATCAATGATGATGATGATGATGGGAGCAG
AGGATGTTGGGAAAGTATTGGTAC

(SEQ ID NO:15)

VII 5′: ACCGTTAGCGGCCGCCACCATGGTGCCACCCAAA
TTGCATGTGCTTTTCTGCC

(SEQ ID NO:16)

VII 3′: TGCCAGTTCTAGATCAATGATGATGATGATGATGATTGT
ATTTCTCTATTCCTGAAGAGTTCTGTAAC

(SEQ ID NO:17)

X 5′: GGTCGACCATGG GGCCGCTACCTGTGTG
X 3′: GGATCCCCCTCCGCTTCCCCCTCCGCTTCCCCCTCC
GCTTCCCCCTCCGTCACACTTGGGCGAGTCCGGCTC
(SPLA2-X-LINKER)

(SEQ ID NO:18)

CAGATCTCAATGGTGATGGTGATGATGGGA TCCCCCTCCGCTTCCCC
(LINKER-6XHIS)

(SEQ ID NO:19)

XIIA 5′: ACCGTTAGCGGCCGCCACCATGGCCCTGCTCTCGCGC
CCCGCGCTCACCC

(SEQ ID NO:20)

XIIA 3′: TGCCAGTTCTAGATCAATGATGATGATGATGATGA
AGATCAGTTTTTTCTTCATAATGACACCTGCA

The primer sequences used for point mutants are listed below.

D47K 5′:

(SEQ ID NO:21)

GACTGGTGCTGCCATGGCCACAAGTGTTGTTACACTCGAGC
D47K 3′:

(SEQ ID NO:22)

GCTCGAGTGTAACAACACTTGTGGCCATGGCAGCACCAGTC
H46N, D47E and Y50F 5′:

(SEQ ID NO:23)

CTGCCATGGCAACGAGTGTTGTTTCACTCGAGCTGAGGA
GGCCGGCTGCAGCC
H46N, D47E and Y50F 3′:

(SEQ ID NO:24)

GGCTGCAGCCGGCCTCCTCAGCTCGAGTGAAACAACACTCGTTGCC
ATGGCAGC

Transfection and Western blot analysis. 293 cells were transfected using calcium phosphate (Promega) and cell culture supernatants were harvested 2 days after transfection and kept at −80° C. Cell culture supernatants were resolved by SDS-PAGE and transferred to a PVDF membrane (Bio-Rad). The membrane was incubated with rabbit polyclonal anti-His antibody (1:1000, Santa Cruz Biotechnology) for 1 hour at room temperature in blocking buffer (Tris-buffered saline, 3% skim milk, 0.5% Triton X-100), followed by washing. The blot was further incubated in blocking buffer with horseradish peroxidase-conjugated goat anti-rabbit IgG (1:5000, Santa Cruz) for 30 min and then washed. Detection was performed with the ECL reagent (Amersham).

Recombinant sPLA2 protein purification. The baculovirus expression vector was made following the standard protocol as described by the company (BD Biosciences and Invitrogen). Briefly, sPLA2-X cDNA and three amino acid mutant sPLA2-X (H46N, D47E, and Y50F) were cloned in pVL1393 (transfer vector) which has an AcMNPV polyhedron enhancer-promoter sequence to drive high expression. The recombinant DNA was verified by sequencing. This plasmid was co-transfected with linearized BD baculoGold™ baculovirus DNA (BD Biosciences and Invitrogen) in Sf9 insect cells to make a recombinant baculovirus. The plaque-purified virus was checked for the presence of the PLA2 gene and was amplified by reinfecting Sf9 insect cells. This high titer recombinant virus was later used to make PLA2 protein in High Five (Hi5) cells.

Culture supernatant from one liter of Hi5 cells infected with above baculovirus was harvested after 48 hr incubation at 27° C. The sample was adjusted to 1×PBS with 10× concentrate and 10 mM imidazole with a 1 M stock, filtered (0.45 μm PES membrane) and applied to a 5 ml HisTrap column (GE Healthcare) and eluted with a 20 column volume linear imidazole gradient to 400 mM and the fractions were analyzed by SDS-PAGE. Final samples were dialyzed to 1×PBS and concentrated using 10K MWCO Amicon Ultra filtration devices (Millipore).

Wild type sPLA2-X and mutant sPLA2-X (D47K) were also expressed in 293 cells and the culture supernatants were applied to a 5 ml HisTrap column and eluted as described above.

sPLA2 enzymatic assay. To measure sPLA2 enzymatic activity in the cell culture supernatant from the indicated DNA-transfected cells, an sPLA2 assay kit (Cayman Chemical) was used according to the manufacturer's recommendation. This assay uses the 1,2-dithio analog of diheptanoyl phosphatidylcholine as a substrate for sPLA2s. Upon hydrolysis of the thio ester bond at the sn-2 position by sPLA2, free thiols are detected using DTNB (5,5-dithio-bis-(2-nitrobenzoic acid)) at 405 nm. The specific activity of sPLA2 was calculated based on the initial slope of the time-dependence of absorption at 405 nm, using an extinction coefficient of ε405 nm=12.8 mM-cm-.

Viruses. HIV-1ADA, HIV-1IIIB, Ebola, and MoMuLV envelope lentivirus expressing luciferase were prepared by transient cotransfection of 293T cells with calcium phosphate (Promega) (28). Briefly, the packaging vector pCMVAR8.2 (7 μg), pHR′CMV-Luc (7 μg) and the envelope expressing vector pSVIII-ADA (10 μg), pRSV-IIIB (10 μg), pVR1012-GP(Z) (50 ng), pNGVL-Env (4070A) (2 μg) or CMV/R-8 kb Influenza H5 (A/Thailand/1 (KAN-1)/2004) HA-wt/h (50 ng) were cotransfected. Supernatants were harvested 72 hours after transfection, filtered with 0.45-μm-pore-size syringe filter, and stored at −80° C.

Ad5-Luciferase virus was made as described previously (2). Wild-type HIV-1BaL and HIV-1MN stocks were prepared in peripheral blood mononuclear cells as previously described (13).

Infection of cells with pseudoviruses and luciferase assay. 30,000 cells were plated into each well of a 48-well dish the day before infection; MAGI-CCR5 for HIVADA, HIVIIIB and MoMuLV, and 786-O cells for Ebola and Ad5. Pseudoviral supernatant (50 to 100 μl) or 1.5×10⁷ viral particles of Ad5 (500/cell) were incubated with sPLA2 or its mutant-transfected cell culture supernatant for 1 hour at 37° C. and added to the target cells. Cells were replenished with fresh medium at 16 to 18 hours postinfection. After 48 hours, cells were lysed in cell lysis buffer (Promega) 80 μl in the plate and 20 μl of cell lysate was used in a luciferase assay with luciferase assay reagent (Promega) according to manufacturer's recommendations. Luciferase assay was measured using Top Count (Packard).

HIV single-round replication assay. To assess the effect of sPLA2-X on live wild-type HIV-1BaL and HIV-1 MN, the virus (p24=100 ng) was incubated with 53 ng of purified sPLA2-X (=400 nmol/min activity) or its mutant (H46N, D47E, and Y50F) for 60 min at 37° C. A3R5 cells (1×10⁶ cells) were added to the above described mixtures for 2 hours allowing infection. Cells were washed and incubated with fresh medium. After 64 hours, cells were stained with FITC-conjugated anti-p24 Gag antibody (KC-57 FITC; Beckman Coulter) and analyzed (13).

Analysis of p24 release from virions by density gradient. Density gradient-purified Ebola pseudoviruses (50 μl) or HIV-BaL (p24=2.5 μg)/sPLA2 mixture was added to the same volume of OptiPrep (Axis-Shield PoC). Density gradient was formed by centrifugation at 421K×g for 3.5 hrs with an NVT100 rotor (Beckman). The collected fractions were weighed and density was calculated. An equal amount of each fraction (20 μl) was separated on a 4-15% SDS-PAGE gel (Bio-Rad), transferred to a PVDF membrane and blotted with human anti-HIV-1 IgG or rabbit anti-p24 Gag serum (Advanced Biotechnologies). Each lane of the Western blot represents one fraction of density gradient.

### Results

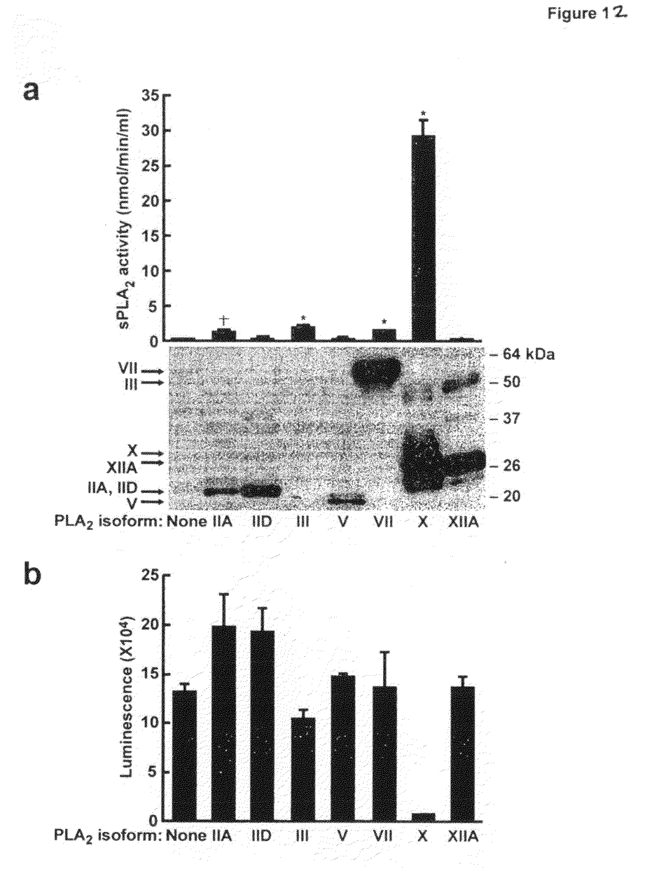

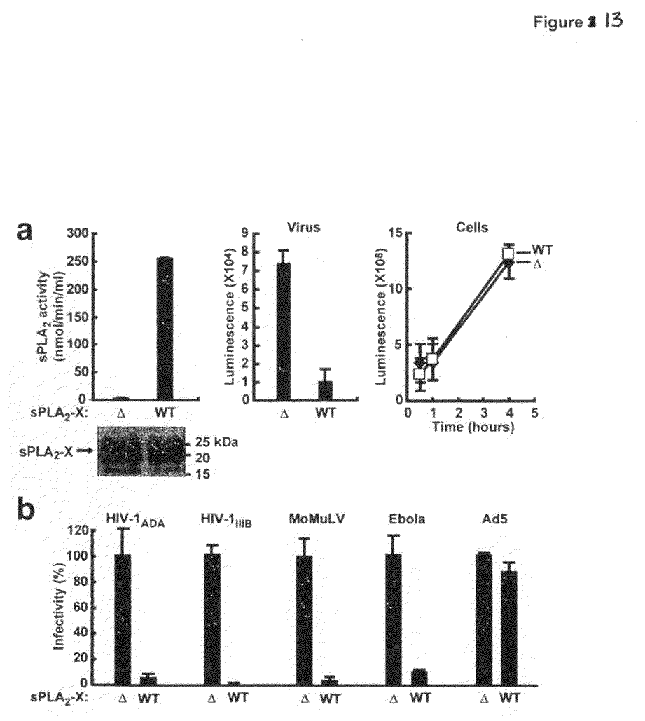

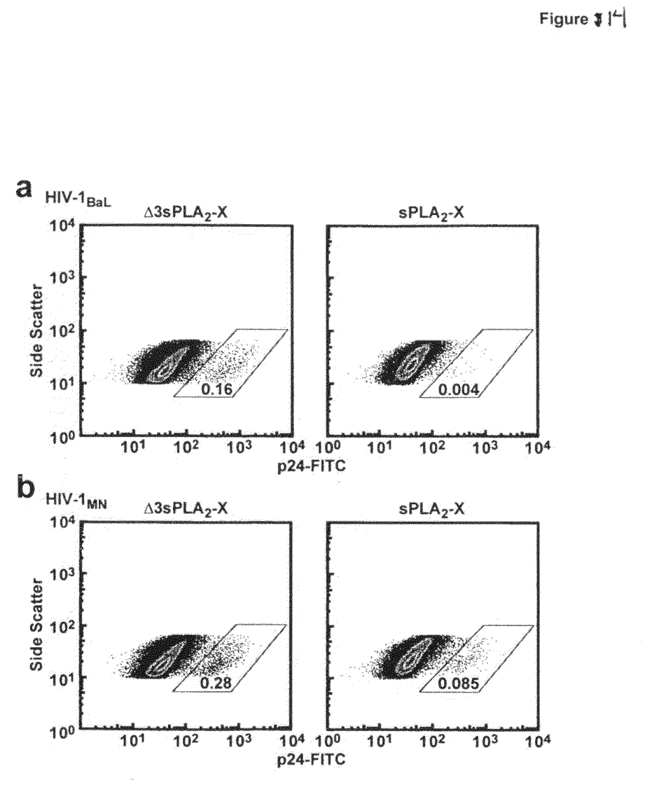

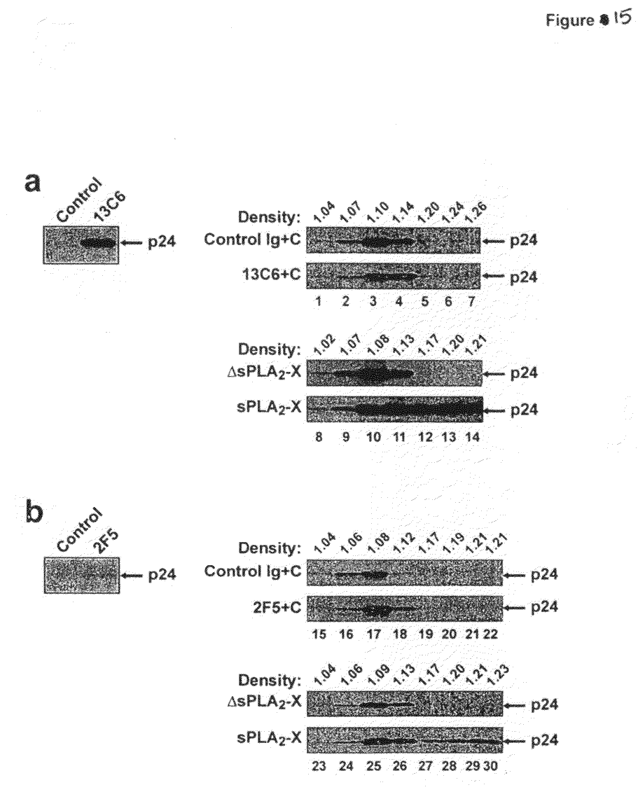

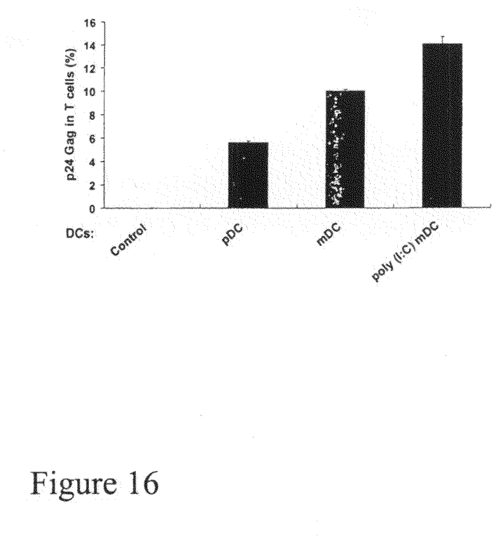

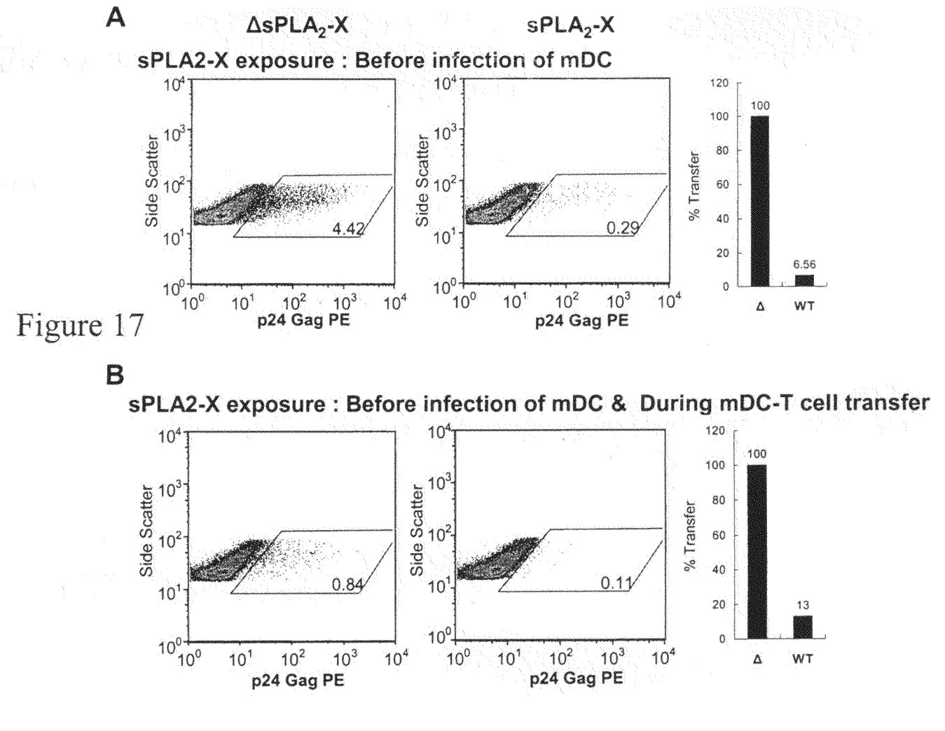

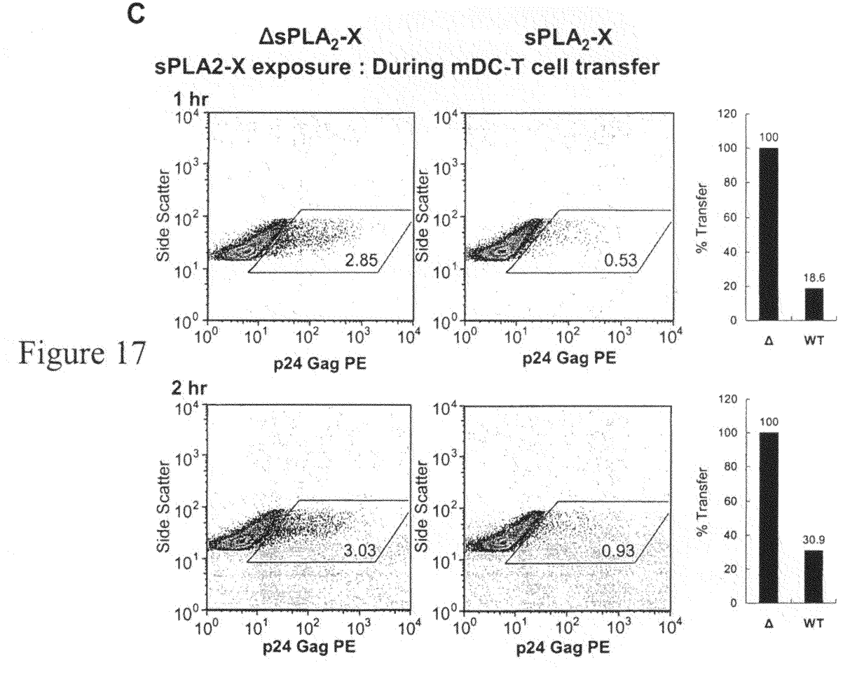

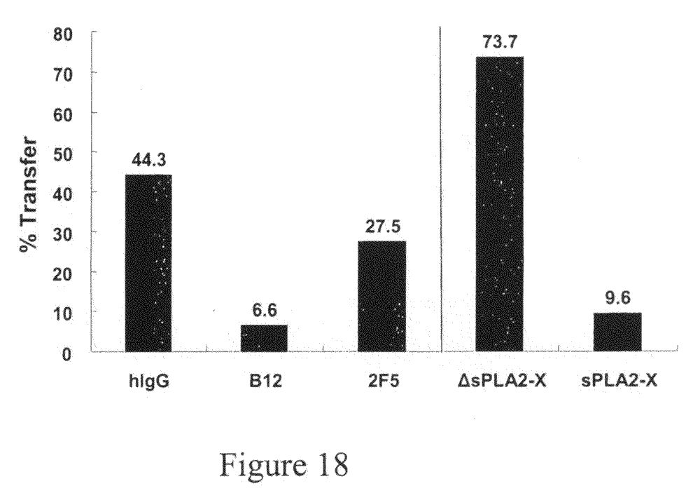

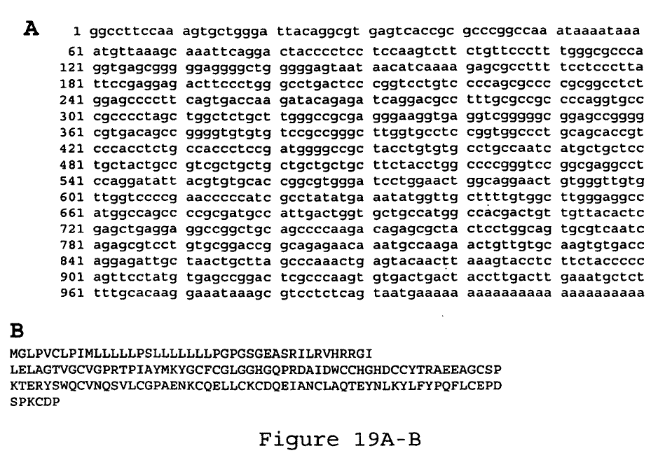

To define the potential of mammalian secretory phospholipase A2 (sPLA2) to confer protection against viral infection, plasmid expression vectors encoding the human group IIA, IID, III, V, VII, X, and XIIA isoforms were prepared and tagged with a COOH-terminal poly-histidine epitope to facilitate detection. When tested for enzymatic activity, group IIA, III, VII and X displayed significant sPLA2 enzymatic activity compared to control supernatants (vector), (IIA; p<0.05, III, VII, and X; p<0.01), with sPLA2-X being the most active (FIG. 12A, upper panel). Expression of each sPLA2 was also confirmed by Western blotting with an anti-His antibody (FIG. 12, lower panel). The antiviral effects of recombinant human sPLA2 cell culture supernatants were tested first by measuring the luciferase reporter gene activity of HIV-1 pseudoviruses on MAGI-CCR5 target cells, a human cervical carcinoma (HeLa) cell line expressing CD4 and co-receptors CXCR4 and CCR5. Among the different sPLA2s, the group X isoform showed marked inhibition of the HIV-1IIIB pseudotype reporter (FIG. 12B). Though sPLA2-X displayed the highest enzymatic activity on this substrate, it was not the highest by protein expression. There is evidence that different sPLA2s have different substrate affinity that may determine their biologic effect (20), suggesting that there is specificity for this effect among the isoforms.

To examine whether catalytic activity was required for its inhibitory effect, wild-type, enzymatically active protein and a catalytically inactive point mutant, D47K, termed ΔsPLA2-X, were generated. Though equivalent amounts of proteins were detected, ΔsPLA2-X showed no catalytic activity (FIG. 13A, left panel). While enzymatically active sPLA2-X markedly inhibited reporter gene expression, similar protein concentrations of inactive ΔsPLA2-X exerted no effect (FIG. 13A, middle panel). sPLA2-X acted primarily through damage to virions because treatment of the target cells of infection did not significantly reduce viral gene transfer (FIG. 13A, right panel).

The specificity of the sPLA2-X antiviral effect was assessed on different viral envelopes expressed on lentiviral vectors, including CXCR4-tropic HIV-1IIIB, CCR5-tropic HIV-1ADA, amphotropic Moloney murine leukemia virus (MoMuLV), Ebola virus glycoprotein (GP), or a non-enveloped viral vector, recombinant adenovirus type 5 (rAd5). Wild-type sPLA2-X showed significant antiviral activity against CCR-5 or CXCR4 tropic HIV Env, amphotropic MoMuLV and Ebola compared to ΔsPLA2-X but did not show significant inhibition of non-enveloped virus, recombinant Ad5 reporter gene expression (FIG. 13B), suggesting that the antiviral activity required the presence of a lipid-containing viral membrane.

The antiviral effect of sPLA2-X was assessed against HIV-1BaL (CCR5-tropic) and HIV-1MN (CXCR4-tropic) stocks produced in peripheral blood mononuclear cells (PBMCs). Virus preparations were incubated with purified sPLA2-X or a different catalytically inactive mutant, A3sPLA2-X (H46N, D47E and Y50F) (9,19) prior to infection of the human T leukemia cell line A3R5, a subline of A3.01 cells (10) expressing both CCR5 and CXCR4. Flow cytometric analysis of intracellular Gag protein was used to assess viral replication. sPLA2-X treatment substantially reduced T cell infection by CCR5-tropic HIV-1BaL (FIG. 14A, right panel) compared to the catalytically inactive A3sPLA2-X (FIG. 14A, left panel). A similar reduction in viral replication was seen when sPLA2-X was incubated with replication-competent CXCR4-tropic HIV-1MN (FIG. 14B), suggesting that this antiviral mechanism is effective against diverse lentiviruses with alternative chemokine receptor specificity.

To understand the mechanism of the sPLA2-X anti-viral effect, the ability of sPLA2-X to lyse virus was examined both in pseudotyped lentiviral vectors and in replication-competent HIV-1BaL derived from peripheral blood mononuclear cells. For the pseudotyped lentiviral vector, Ebola GP pseudotypes were analyzed first, using gradient-purified virions. The presence of p24 Gag in different gradient fractions was first confirmed by immunoprecipitation followed by Western blotting, with peak activity at a density of 1.10 (FIG. 15A, right panel, lane 3). Analysis of virions from this purified fraction revealed reactivity with monoclonal antibody 13C6, known to bind Ebola GP on virions (27) (FIG. 15A, left panel). This antibody of IgG2a subtype has been shown to fix complement (27). Gradient-purified pseudotyped virions were treated with control mouse IgG or 13C6 plus mouse complement. Though virions reacted with this antibody and are able to fix complement, no release of p24 Gag was detected as shown by re-fractionation through the density gradient (FIG. 15A, right panel, lanes 5-7). In contrast, treatment with sPLA2-X, but not ΔsPLA2-X (D47K), caused Gag release when these virions were re-fractionated through a density gradient (FIG. 15A, right lower panel, sPLA2-X vs. ΔsPLA2-X, lanes 12-14). A similar effect was observed with the 2F5 broadly neutralizing human monoclonal antibody of IgG1 subtype that binds HIV-1BaL (FIG. 15B), confirming its effect on native virus.

### Discussion

In this study, the ability of sPLA2s to inhibit HIV-1 replication has been evaluated. We find that sPLA2-X displays antiviral activity against diverse lentiviruses by degradation of the viral membrane. sPLA2-X inhibits replication of both CXCR4- and CCR5-tropic HIV-1 in primary human CD4+ cells. This effect was observed despite the resistance of virus preparations to lysis by antibody-mediated complement activation, suggesting that this mechanism acts in cases where the acquired immune response is ineffective.

### Example 6

### Investigation of the Role of sPLA2s in CCR5-Tropic HIV-1_{BaL} Transfer from Myeloid Dendritic Cells to CD4+ T Cells

For the following experiments, HIV-1_{BaL} stock was prepared in peripheral blood mononuclear cells.

HIV-1_{BaL} transfer from dendritic cells to CD4⁺ T cells: Plasmacytoid dendritic cells (pDC), myeloid dendritic cells (mDC) and poly (I-C) treated mDCs (3×10⁴ cells) isolated from elutriated monocytes of a single donor were either mock infected (control) or infected with HIV-1BaL (50 ng of p24) for 2 hrs and washed. Primary PHA-IL-2-stimulated autologous CD4+ T cells (1.25×10⁵ cells) were added to both mock-infected and HIV-1-infected DCs and incubated for another 72 hrs. p24 Gag in CD3⁺ cells was then analyzed by flow cytometry (FIG. 16).

Effect of sPLA2-X exposure on HIV-1 BaL trans-infection from mDC to CD4+ T cells: Wild-type HIV-1_{BaL} (30 ng of p24) was added to either sPLA2-X (100 nmol/min activity) or equivalent amount (by weight) of catalytically inactive D47K mutant of sPLA2-X (ΔsPLA2-X) for 60 min before infection of poly (I:C)-treated mDCs (4×10⁴ cells each) for 2 hrs (A and B). Alternatively, viruses were directly used to infect poly (I:C) treated mDCs (C). mDCs were washed five times to remove virus and incubated with autologous CD4⁺ T cells alone (1.2×10⁵ cells each) (A) or treated with sPLA2-X (100 nmol/min activity) or equivalent amount of ΔsPLA2-X and CD4⁺ T cells (1.2×10⁵ cells each) (B and C) for 2 hrs. Cells were washed three times and cultured for additional 72 hrs. p24 Gag in CD3⁺ cells was then analyzed by flow cytometry. % transfer was shown in the right panel (Δ=ΔsPLA2-X, and WT=sPLA2-X) (FIG. 17).

Comparison of the effects of sPLA2-X and neutralizing antibodies on HIV-1_{BaL} trans-infection from mDCs to CD4⁺ T cells: Poly (I:C)-treated mDCs were infected with HIV-1_{BaL} for 2 hrs, washed five times, and incubated with human IgG (hIgG), B12, 2F5 (each 50 μg/ml), sPLA2-X (100 nmol/min activity) or equivalent amount of catalytically inactive D47K mutant of sPLA2-X (ΔsPLA2-X) and primary PHA-IL-2 stimulated autologous CD4⁺ T cells for 2 hrs. Cells were washed 3 times and cultured for another 72 hrs. p24 Gag in CD3+ cells was assayed by flow cytometry. % transfer was defined as the number of p24-Gag positive cells compared to the number in control wells (no antibody or no sPLA2-X) during transfer (FIG. 18)

### CONCLUSION

The experiments set forth in Example 6 indicate that sPLA2-X neutralizes HIV-1_{BaL} transfer from mDC to CD4⁺ T cells in vitro. Moreover, the results demonstrate that the inhibitory function is more efficient than 2F5 neutralizing antibody.

The results further indicate that sPLA2-X limits viral replication and reduces the incidence of productive replication at sites of primary infection.

### REFERENCES

1. Aasa-Chapman, M. M., S. Holuigue, K. Aubin, M. Wong, N. A. Jones, D. Cornforth, P. Pellegrino, P. Newton, I. Williams, P. Borrow, and A. McKnight. 2005. Detection of antibody-dependent complement-mediated inactivation of both autologous and heterologous virus in primary human immunodeficiency virus type I infection. J. Virol. 79:2823-2830.
2. Aoki, K., C. Barker, X. Danthinne, M. J. Imperiale, and G. J. Nabel.
1999. Efficient generation of recombinant adenoviral vectors by Cre-lox recombination in vitro. Mol. Med. 5:224-231.
3. Berg, O. G., M. H. Gelb, M. D. Tsai, and M. K. Jain. 2001. Interfacial enzymology: the secreted phospholipase A(2)-paradigm. Chem. Rev 101:2613-2654.
4. Bezzine, S., R. S. Koduri, E. Valentin, M. Murakami, I. Kudo, F. Ghomashchi, M. Sadilek, G. Lambeau, and M. H. Gelb. 2000. Exogenously added human group X secreted phospholipase A(2) but not the group IB, IIA, and V enzymes efficiently release arachidonic acid from adherent mammalian cells. J. Biol. Chem. 275:3179-3191.
5. Brugger, B., B. Glass, P. Haberkant, I. Leibrecht, F. T. Wieland, and H. G. Krausslich. 2006. The HIV lipidome: a raft with an unusual composition. Proc. Natl. Acad. Sci. U.S.A 103:2641-2646.
6. Fenard, D., G. Lambeau, E. Valentin, J. C. Lefebvre, M. Lazdunski, and A. Doglio. 1999. Secreted phospholipases A(2), a new class of HIV inhibitors that block virus entry into host cells. J. Clin. Invest 104:611-618.
7. Gronroos, J. O., V. J. Laine, M. J. Janssen, M. R. Egmond, and T. J. Nevalainen. 2001. Bactericidal properties of group IIA and group V phospholipases A2. J. Immunol. 166:4029-4034.
8. Hanasaki, K., T. Ono, A. Saiga, Y. Morioka, M. Ikeda, K. Kawamoto, K. Higashino, K. Nakano, K. Yamada, J. Ishizaki, and H. Arita. 1999. Purified group X secretory phospholipase A(2) induced prominent release of arachidonic acid from human myeloid leukemia cells. J. Biol. Chem. 274:34203-34211.
9. Janssen, M. J., W. A. van de Wiel, S. H. Beiboer, M. D. van Kampen, H. M. Verheij, A. J. Slotboom, and M. R. Egmond. 1999. Catalytic role of the active site histidine of porcine pancreatic phospholipase A2 probed by the variants H48Q, H48N and H48K. Protein Eng 12:497-503.
10. Kim, J. H., P. Pitisuttithum, C. Kamboonruang, T. Chuenchitra, J. Mascola, S. S. Frankel, M. S. DeSouza, V. Polonis, R. McLinden, A. Sambor, A. E. Brown, B. Phonrat, K. Rungruengthanakit, A. M. Duliege, M. L. Robb, J. McNeil, and D. L. Birx. 2003. Specific antibody responses to vaccination with bivalent CM235/SF2 gp 120: detection of homologous and heterologous neutralizing antibody to subtype E (CRF01.AE) HIV type 1. AIDS Res. Hum. Retroviruses 19:807-816.
11. Koduri, R. S., J. O. Gronroos, V. J. Laine, C. Le Calvez, G. Lambeau, T. J. Nevalainen, and M. H. Gelb. 2002. Bactericidal properties of human and murine groups I, II, V, X, and XII secreted phospholipases A(2). J. Biol. Chem. 277:5849-5857.
12. Laine, V. J., D. S. Grass, and T. J. Nevalainen. 2000. Resistance of transgenic mice expressing human group II phospholipase A2 to Escherichia coli infection. Infect. Immun. 68:87-92.
13. Mascola, J. R., M. K. Louder, C. Winter, R. Prabhakara, S. C. DeRosa, D. C. Douek, B. J. Hill, D. Gabuzda, and M. Roederer. 2002. Human immunodeficiency virus type I neutralization measured by flow cytometric quantitation of single-round infection of primary human T cells. J. Virol. 76:4810-4821.
14. Masuda, S., M. Murakami, Y. Takanezawa, J. Aoki, H. Arai, Y. Ishikawa, T. Ishii, M. Arioka, and I. Kudo. 2005. Neuronal expression and neuritogenic action of group X secreted phospholipase A2. J. Biol. Chem. 280:23203-23214.
15. Mitsuishi, M., S. Masuda, I. Kudo, and M. Murakami. 2006. Group V and X secretory phospholipase A2 prevents adenoviral infection in mammalian cells. Biochem. J. 393:97-106.
16. Mounier, C. M., F. Ghomashchi, M. R. Lindsay, S. James, A. G. Singer, R. G. Parton, and M. H. Gelb. 2004. Arachidonic acid release from mammalian cells transfected with human groups IIA and X secreted phospholipase A(2) occurs predominantly during the secretory process and with the involvement of cytosolic phospholipase A(2)-alpha. J. Biol. Chem. 279:25024-25038.
17. Murakami, M., T. Kambe, S. Shimbara, K. Higashino, K. Hanasaki, H. Arita, M. Horiguchi, M. Arita, H. Arai, K. Inoue, and 1. Kudo. 1999. Different functional aspects of the group II subfamily (Types IIA and V) and type X secretory phospholipase A(2)s in regulating arachidonic acid release and prostaglandin generation. Implications of cyclooxygenase-2 induction and phospholipid scramblase-mediated cellular membrane perturbation. J. Biol. Chem. 274:31435-31444.
18. Murakami, M. and I. Kudo. 2001. Diversity and regulatory functions of mammalian secretory phospholipase A2s. Adv. Immunol. 77:163-194.
19. Sekar, K., R. Biswas, Y. Li, M. Tsai, and M. Sundaralingam. 1999. Structures of the catalytic site mutants D99A and H48Q and the calcium-loop mutant D49E of phospholipase A2. Acta Crystallogr. D. Biol. Crystallogr. 55:443-447.
20. Singer, A. G., F. Ghomashchi, C. Le Calvez, J. Bollinger, S. Bezzine, M. Rouault, M. Sadilek, E. Nguyen, M. Lazdunski, G. Lambeau, and M. H. Gelb. 2002. Interfacial kinetic and binding properties of the complete set of human and mouse groups I, II, V, X, and XII secreted phospholipases A2. J. Biol. Chem. 277:48535-48549.
21. Stoiber, H., M. Pruenster, C. G. Ammann, and M. P. Dierich. 2005. Complement-opsonized HIV: the free rider on its way to infection. Mol. Immunol. 42:153-160.
22. Sullivan, B. L., E. J. Knopoff, M. Saifuddin, D. M. Takefinan, M. N. Saarloos, B. E. Sha, and G. T. Spear. 1996. Susceptibility of HIV-1 plasma virus to complement-mediated lysis. Evidence for a role in clearance of virus in vivo. J. Immunol. 157:1791-1798.
23. Valentin, E., A. G. Singer, F. Ghomashchi, M. Lazdunski, M. H. Gelb, and G. Lambeau. 2000. Cloning and recombinant expression of human group IIF-secreted phospholipase A(2). Biochem. Biophys. Res. Commun. 279:223-228.
24. Verheij, H. M., A. J. Slotboom, and G. H. de Haas. 1981. Structure and function of phospholipase A2. Rev Physiol Biochem. Pharmacol. 91:91-203.
25. Verity, E. E., L. A. Williams, D. N. Haddad, V. Choy, C. O'Loughlin, C. Chatfield, N. K. Saksena, A. Cunningham, F. Gelder, and D. A. McPhee. 2006. Broad neutralization and complement-mediated lysis of HIV-1 by PEHRG214, a novel caprine anti-HIV-1 polyclonal antibody. AIDS 20:505-515.
26. Weinrauch, Y., P. Elsbach, L. M. Madsen, A. Foreman, and J. Weiss. 1996. The potent anti-Staphylococcus aureus activity of a sterile rabbit inflammatory fluid is due to a 14-kD phospholipase A2. J. Clin. Invest 97:250-257.
27. Wilson, J. A., M. Hevey, R. Bakken, S. Guest, M. Bray, A. L. Schmaljohn, and M. K. Hart. 2000. Epitopes involved in antibody-mediated protection from Ebola virus. Science 287:1664-1666.
28. Yang, Z.-Y., Y. Huang, L. Ganesh, K. Leung, W.-P. Kong, O, Schwartz, K. Subbarao, and G. J. Nabel. 2004. pH-dependent entry of Severe Acute Respiratory Syndrome coronavirus is mediated by the Spike glycoprotein and enhanced by dendritic cell transfer through DC-SIGN. J. Virol. 78:5642-5650.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A polypeptide comprising a phospholipase polypeptide, or biologically active fragment thereof, and a viral binding polypeptide.

2. The polypeptide of claim 1, further comprising a polypeptide tinker.

3. The polypeptide of claim 1, wherein the phospholipase polypeptide is a mammalian phospholipase.

4-14. (canceled)

15. The polypeptide of claim 1, wherein the viral binding polypeptide is a DC-SIGN polypeptide, or biologically active fragment thereof.

16. The polypeptide of claim 15, wherein the DC-SIGN polypeptide or biologically active fragment thereof, has the sequence as set forth in SEQ ID NO:3.

17. The polypeptide of claim 16, wherein the DC-SIGN polypeptide or biologically active fragment thereof, is at least 90% identical to the sequence set forth as SEQ ID NO:3.

18. The polypeptide of claim 2, wherein the polypeptide linker is comprised of glycine and serine amino acid residues.

19. A polypeptide comprising phospholipase A2, or a biologically active fragment thereof, and DC-SIGN polypeptide connected by a peptide linker.

20. The polypeptide of claim 19 having the sequence set forth as SEQ ID NO:5.

21. A polynucleotide encoding a polypeptide comprising a phospholipase polypeptide, or biologically active fragment thereof, and a viral binding polypeptide.

22. (canceled)

23. The polynucleotide of claim 21, wherein the phospholipase polypeptide is a mammalian phospholipase.

24. The polynucleotide of claim 23, wherein the mammalian phospholipase is a human phospholipase.

25-40. (canceled)

41. A vector comprising the nucleic acid sequence of claim 21.

42. (canceled)

43. The vector of claim 41 wherein the expression vector is a CMV/R expression vector.

44. A host cell comprising the expression vector of claim 41.

45. (canceled)

46. A pharmaceutical composition comprising an effective amount of a polypeptide according to claim 1 and a pharmaceutically acceptable carrier.

47. A pharmaceutical composition comprising an effective amount of a polynucleotide according to claim 21 and a pharmaceutically acceptable carrier.

48. A method of treating a subject having a viral infection comprising:
administering to the subject an effective amount of a polypeptide of claim 1;
thereby treating a subject having a viral infection.

49. The method of claim 48, wherein the viral infection is caused by an enveloped virus.

50. The method of claim 49, wherein the enveloped virus is selected from the group consisting of a Herpesviridae virus, a Poxyiridae virus, a Hepadnaviridae virus, a Togaviridae virus, a Flaviviridae virus, a Coronaviridae virus; a Paramyxoviridae virus, a Bunyaviridae virus, a Rhabdoviridae virus, a Filoviridae virus, an Orthomyxoviridae virus, an Arenaviridae virus, and a Retroviridae virus.

51. The method of claim 49, wherein the enveloped virus is selected from the group consisting of HIV, Hepatitis, Amphovirus, Marburg, Ebola, Herpes Simplex Virus (HSV) Type 1, Herpes Simplex Virus Type 2, Vesicular Stomatitis Virus (VSV), Visna Virus (VV), Measles Virus (MV), and SARS.

52. A method of preventing an infection in a subject comprising:
administering to the subject an effective amount of a polypeptide of claim 1;
thereby preventing a viral infection in a subject.

53-78. (canceled)

79. A method of treating or preventing a subject having a viral infection comprising:
administering to the subject an effective amount of a polynucleotide of claim 21;
thereby treating or preventing a subject having a viral infection.
